(19) United States
(12) Patent Application Publication
(10) Pub. No.: US 2019/0209007 A1
(43) Pub. Date: Jul. 11, 2019

(54) WEARABLE MEDICAL DEVICE AND SYSTEMS DERIVED THEREFROM

(71) Applicants: Paul S. D'URSO, Richmond (AU); Philip LEWIS, Richmond (AU)

(72) Inventors: Paul S. D'URSO, Richmond (AU); Philip LEWIS, Richmond (AU)

(21) Appl. No.: 16/325,765

(22) PCT Filed: Aug. 18, 2017
(86) PCT No.: PCT/AU2017/050883

§ 371 Date: Feb. 15, 2019

(30) Foreign Application Priority Data
Aug. 18, 2016 (AU) 2016903275

(51) Int. Cl. A61B 5/00; A61N 1/05; A61B 5/01; A61B 5/11

(52) U.S. Cl. CPC A61B 5/002; A61N 1/0529; A61B 5/01; A61B 5/6804; A61B 5/0008; A61B 5/6806; A61B 5/1112; A61B 5/6868; A61B 5/6803

(57) ABSTRACT

Wearable medical devices and systems comprising the devices are provided. The devices and systems find use in monitoring and/or modulating the health of a wearer. The wearable devices may be personalized to conform to the external surface anatomy of the wearer and comprise one or more functional elements. The functional elements are positioned within the wearable device so as to maximize alignment or interaction with functional elements in one or more implanted medical devices or one or more internal anatomical features. The devices and systems find use in the delivery of health care.

### FIELD

The present disclosure relates to wearable medical devices and to systems including such wearable devices. The disclosure particularly relates to wearer specific wearable medical devices, wearer specific implanted devices and to systems derived therefrom. The devices and systems find use in the delivery of healthcare.

### BACKGROUND

The field of implantable medical devices is evolving rapidly, as technological developments provide improved longevity, an expanded scope of functionality and continued miniaturization. These developments also allow for wider applications in the treatment of disease and a reduction in the prevalence of unwanted treatment side-effects or complications.

However, an ongoing technology challenge in the development of fully-implanted medical devices is incorporating sufficient energy storage capacity such that the device can be independently powered for an appropriate length of time, without requiring replacement. Additional challenges associated with the operation of such devices include bidirectional communication between the implant and the external environment.

With the increasing complexity of implantable devices, or, in the treatment of certain disease states, the ability of implanted medical devices to perform their intended function without the presence of a component that is external to the body becomes limited or impossible. An example of this limitation may be found in the concept of the “bionic eye” or bionic vision implant. In many embodiments of bionic vision implants, the implantable component of the system comprises an array of electrodes that provide electrical stimuli to the visual pathways, and associated electronics which themselves provides electrical stimulus pulses to the electrodes. These devices, in most cases, rely on the use of an external camera device to provide image data to the implanted component of the system. This provides an example of a device wherein its intended function would not be possible without the inclusion of an integral, external component that allows the system as a whole to perform its intended function. In a general sense, such devices could be considered a system, wherein the internal and external components both perform unique functions that are required for the system to operate correctly.

Patients suffering stroke, head injury, epilepsy, brain tumours, and other neurological diseases or disorders that require a neurosurgical procedure will often require the removal of a portion of skull bone to permit surgical access to the brain. In some cases, the removed portion of skull bone will not be replaced immediately. Rather, there will be a persistent defect left in the skull for a period of time, determined by the treating surgeon and/or neurologist, until the patient has recovered to the point that the skull defect may be restored. In other cases, the bone will be replaced, or the portion of skull bone removed will be small enough that no restoration of the defect is required.

The extent of the bone removal is typically such that the patient is required to wear protective headgear, particularly while ambulant, to provide protection to the brain exposed by the defect in the skull. Typically, such protective headgear is generic, being a commercially-available, consumer-grade device that is not customised to fit the wearer, beyond the usual methods of customization to fit head size and shape that are provided by consumer-grade protective headgear.

U.S. Pat. No. 5,549,678 to Prostkoff discloses patient-specific headgear intended to provide an aesthetically-pleasing, impact-resistant covering for a skull defect, and a means of holding this covering onto the head of the wearer. Prostkoff discloses the forming of a thermoplastic sheet by placement of heated, softened thermoplastic material onto the side of the skull opposite the defect, after which the material is placed over the defect itself for final shaping once the material has hardened slightly.

U.S. Pat. No. 9,232,827 to Penn discloses a helmet to protect a patient's head after, for example, cranial surgery. Specifically, Penn discloses a protective helmet intended to provide protection to the brain of a craniectomy patient, and which provides space and access holes for monitoring sensors. Penn discloses the manufacture of generic prefabricated helmets or patient-specific helmets, using the techniques of subtractive manufacturing (using a multi-axis router) based on patient imaging data, or moulding thermoplastic over a generic human head model, to produce a positive mould for vacuum forming or thermoforming of the final device.

U.S. Pat. No. 6,798,392 to Hartwell et al discloses a helmet including integrated electronics providing safety and convenience features. The helmet includes a module which can monitor the characteristics of the user and the user's environment, communicating this information to the user and others. Hartwell et al disclose selected characteristics including location, instantaneous speed and distance traveled. Hartwell et al also disclose how the helmet may provide the ability to communicate with others using a mobile communication device.

US 2008/0319281 A1 to Aarts discloses a portable device for detecting a medical condition, such as an epileptic seizure. Aarts discloses a device incorporating at least one sensor, sensing a physical phenomenon in a patient's body, wherein the means for affixing the sensor includes headgear that itself carries the sensor and at least one processor. Aarts also discloses a device comprising at least one motion detection and acceleration detection device, heartbeat detection device and electroencephalography recording device. The purpose of the device disclosed by Aarts is to detect epileptic seizures and physiological and physical phenomena.

Despite the above advances in the art it would be desirable to provide improved wearable medical devices, improved implantable medical devices and systems comprising such devices.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

### SUMMARY

### Personalized Wearable Medical Devices

In a first aspect there is provided a wearable medical device, said device comprising one or more surfaces, said one or more surfaces having at least one region contoured to substantially match at least one contour of an external anatomical surface of a wearer, said device comprising one or more functional elements.

The one or more contoured surface regions of the wearable medical device may comprise a part or a whole of any one or more of the surfaces of the device that are, in use, in contact with the external anatomical surface of the wearer.

The one or more contoured surface regions of the wearable medical device may be designed so that the device closely fits an external anatomical surface of the wearer.

By ‘substantially match’ it may mean that the contoured surface region of the wearable medical device fits an external anatomical surface of a wearer without kinks or overlaps.

The one or more contoured surface regions of the wearable medical device may be determined by wearer specific computer imaging data or other processes.

The shape and dimensions of the one or more contoured surface regions of the wearable medical device may be determined using computer software via automated processes, or via human input or by a combination of both

The shape and dimensions of the one or more contoured surface regions of the device may be determined entirely by human input without the use of a computer.

The shape and dimensions of the one or more contoured surface regions of the wearable medical device may be determined by using the techniques of, for example, hand crafting or sculpting, forming or moulding.

In one embodiment the one or more contoured surface regions of the wearable medical device may substantially match the contours of a specific region of the external anatomical surface of the wearer. For example, to substantially match the external anatomical surface of all or part of the head, neck, chest, abdomen, pelvis or limbs of the wearer.

In one embodiment the one or more contoured surface regions of the wearable medical device may substantially match the contours of more than one region of the external anatomical surface of the wearer. For example, the wearable medical device may be a body suit or any part thereof.

The wearable medical device may be a full body suit, a shirt, a vest, a head band or a head cap.

The wearable medical device may find use in the monitoring and/or modulation of the health of the wearer.

The one or more functional elements may be positioned and/or orientated within the wearable medical device so that, in use, they substantially align with specific regions of the external anatomical surface of the wearer.

Examples of functional elements include, but are not limited to, sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures or encasing structures.

The functional elements in the wearable medical device may be of any three dimensional shape. Preferred three dimensional shapes include, for example, a sphere, a spheroid, a cube, a rectangular prism, a prism, a triangular prism, a cylinder, a cone, a pyramid, or combinations thereof.

The functional elements may provide means for one or more of sensing, communicating, actuating, delivering or receiving information, delivering or receiving energy, or generating energy.

The means for sensing may be one or more of chemical, electrical, physical, optical or magnetic.

The means for stimulating may be one or more of chemical, electrical, physical, optical or magnetic.

The means for actuating may be one or more of pressure, vacuum or physical deformation.

The means for energy generation may be, for example, based on physical movement of the wearer or based on other movements.

The functional elements may provide means for monitoring and/or measuring wearer specific parameters and/or non-wearer specific parameters.

Wearer specific parameters include, but are not limited to, temperature, pressure, electrophysiological changes or the concentration and/or nature of one or more chemical species.

Non-wearer specific parameters include, but are not limited to, temperature, pressure, light intensity, electromagnetic radiation, sound, or the concentration and/or nature of one or more chemical species.

The one or more functional elements may provide means for stimulating the wearer.

The one or more functional elements may be in communication with one or more implanted devices. The one or more functional elements may deliver electrical power to, for example, an implanted device.

In one embodiment the functional elements may sense and quantify environmental parameters, for example, light, sound, electromagnetic radiation, thermal radiation or gravitational forces.

In one embodiment the functional elements may comprise GPS and satellite communication systems.

In one embodiment the functional elements may allow remote monitoring and feedback.

The one or more functional elements may be in communication with each other. The one or more functional elements may form a communication network.

The one or more functional elements may comprise one or more electronic components.

The one or more electronic components may provide means for one or more of sensing, stimulating, communicating, actuating, delivering or receiving information, delivering or receiving energy, or generating energy.

In one embodiment the wearable medical device may comprise more than one functional element positioned within different regions of the device. For example, the wearable medical device may be a body suit or parts thereof.

According to the first aspect of the present disclosure the shape and dimensions of the one or more contoured surface regions of the wearable medical device may be determined using computer software via automated processes.

For example computer imaging data may be obtained from one or more imaging devices, that is, any device or devices either singly or in combination that can capture and represent, in digital form, the external anatomy of the human body (the external anatomical data) of the wearer. Examples of such devices include, but are not limited to, Computed Tomography, Magnetic Resonance Imaging, Ultrasound, one or more lasers, one or more digital cameras, and medical ultrasound.

When positioned on a wearer's head the wearable device may be headgear. Examples of headgear include, but are not limited to, a helmet, a cap, and a headband.

Using the anatomical data, the wearable device may be designed to fit the intended wearer by either a person skilled in the use of three dimensional design software, or by using a set of processes automated in software.

In one embodiment the wearable device may comprise a sheet of, for example, a thermoplastic material. The wearable device may comprise a thin sheet. The wearable device may comprise an impact-resistant thermoplastic. The wearable device may be aesthetically-pleasing.

The wearable device, for example sheet, may have a thickness between about 0.05 mm and 10 mm, or between about 0.1 mm and about 5 mm, or between about 0.2 mm and about 2 mm. The wearable device, for example sheet, may have a thickness less than 10 mm, or less than 5 mm, or less than 2 mm, or less than 1 mm.

The wearable device, for example sheet, may be flexible. The device, for example sheet, may be elastic. The wearable device, for example sheet, may be curved.

Where the wearable device is headgear, the device, for example sheet, may be shaped to conform to the temporal, parietal, frontal or occipital bones of a cranium, or combinations and variations thereof. The device, for example sheet, may be shaped to conform to the left or right sides of the cranium.

The wearable device, for example sheet, may be manufactured as a pre-shaped sheet. It may be pre-shaped to conform to the surface contours of the internal or external anatomy of the wearer. That is, the device, for example sheet, is preferably not a flat sheet that has been curved in a single dimension, but rather a sheet that has been manufactured to substantially conform to the contours of internal or external wearer anatomy. For example, the device, for example sheet, may be substantially dome shaped so as to conform to a respective dome shaped contour of wearer anatomy. The device, for example sheet, and the contour of external wearer anatomy may have a substantially hand and glove relationship.

Where the wearable device is headgear, it may be based on the form of a conventional protective helmet such as might be worn by cyclists or other sportspeople, for example rugby or Australian Rules Football players or North American Football (Gridiron) players. In this embodiment, a support structure intended to distribute the force of any impact applied to the helmet to the skull bone around the skull defect may be designed using the anatomical data. A preferred method of manufacturing this support structure would be using additive manufacturing, however it could also be manufactured using subtractive techniques, injection moulding and the like.

In one embodiment, one or more elements of a helmet structure, including inner shells for providing optimal anatomical contour, and outer shells for providing protection, or both, may be designed using the anatomical data of the wearer, and manufactured using additive manufacturing techniques to provide optimal fit, comfort and therefore protection to the brain of a patient, for example a craniectomy patient.

In one embodiment, the wearable device anatomically conforms precisely to the contour of the skull of the intended wearer. The contour of the skull is obtained and determined by using data provided from one or more imaging devices.

The wearable device may, at least in part, be manufactured using additive manufacturing. It will be appreciated that the practice of applying a heated thermoplastic directly to the external anatomy of the wearer may result in injury and/or suboptimal precision of the contour, with the added potential for discomfort of the wearer and/or suboptimal protection of the anatomy. Advantageously, additive manufacturing provides the ideal method of manufacturing a wearable device based on the anatomical data provided from the one or more imaging devices.

In one embodiment the wearable device may be a full body suit or any part thereof. For example, the wearable device may be a suit or any part thereof manufactured from a suitable elastic material that closely fits the wearer's external anatomy. Non-limiting elastic materials include Spandex®, Lycra® and the like.

The suit, or any part thereof, may comprise one or more functional elements as herein disclosed suitably positioned and/or orientated within the suit, or parts thereof, so as to substantially align with specific surface regions of the external anatomy of the wearer. Accordingly, optimum and advantageous operation of the functional elements may result.

According to the first aspect of the present disclosure the one or more functional elements may be in communication with one or more remote devices. The one or more functional elements may form a network comprising one or more remote devices. Remote devices include, but are not limited to, mobile communication devices such as phones, remote computer servers and the like.

The one or more functional elements may be physically connected to one or more functional elements in one or more devices implanted in the wearer.

The implanted devices include, but are not limited to, sensory devices, neurological devices, cardiovascular devices, orthopaedic devices, contraceptive devices and cosmetic devices.

The connection may be via suitable male and female connectors. The connection may be temporary or permanent.

The one or more functional elements may be in wireless communication with an internally implanted device.

In a second aspect there is provided a wearable medical device, said device comprising one or more functional elements, wherein at least one of said functional elements is positioned within said wearable medical device such that, in use, the at least one functional element aligns with an internal anatomical feature of the wearer.

Internal anatomical features of the wearer include, but are not limited to, vascular features, physical features, functional features and electrical features or combinations thereof. Vascular features, such as vascular topography, may be determined by, for example, vascular mapping. Electrical features may be determined by, for example, electroencephalography.

By aligns it is meant that the position of the at least one functional element in the device is such that its interaction with a target internal feature of the wearer is substantially maximized.

Examples of functional elements include, but are not limited to, sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures and encasing structures.

The functional elements in the wearable medical device may be of any three dimensional shape. Preferred three dimensional shapes include, for example, a sphere, a spheroid, a cube, a rectangular prism, a prism, a triangular prism, a cylinder, a cone, a pyramid, or combinations thereof.

The functional elements may provide means for one or more of sensing, communicating, actuating, delivering or receiving information, delivering or receiving energy, or generating energy.

The means for sensing may be one or more of chemical, electrical, physical, optical or magnetic.

The means for stimulating may be one or more of chemical, electrical, physical, optical or magnetic.

The means for actuating may be one or more of pressure, vacuum or physical deformation.

The means for energy generation may be, for example, based on physical movement of the wearer or based on other movements.

The functional elements may provide means for monitoring and/or measuring wearer specific parameters and/or non-wearer specific parameters.

Wearer specific parameters include, but are not limited to, temperature, pressure, electrophysiological changes or the concentration and/or nature of one or more chemical species.

Non-wearer specific parameters include, but are not limited to, temperature, pressure, light intensity, electromagnetic radiation, sound, or the concentration and/or nature of one or more chemical species.

The one or more functional elements may provide means for stimulating the wearer.

The one or more functional elements may be in communication with one or more implanted devices. The one or more functional elements may deliver electrical power to, for example, an implanted device.

In one embodiment the functional elements may sense and quantify environmental parameters, for example, light, sound, electromagnetic radiation, thermal radiation or gravitational forces.

In one embodiment the functional elements may comprise GPS and satellite communication systems.

In one embodiment the functional elements may allow remote monitoring and feedback.

The one or more functional elements may be in communication with each other. The one or more functional elements may form or be part of a communication network.

The one or more functional elements may comprise one or more electronic components.

The one or more electronic components may provide means for one or more of sensing, stimulating, communicating, actuating, delivering or receiving information, delivering or receiving energy, or generating energy.

In one embodiment the wearable medical device may comprise more than one functional element positioned within different regions of the device. For example, the wearable medical device may be a body suit or parts thereof.

According to the second aspect of the present disclosure the positioning of the one or more functional elements in the wearable medical device may be determined using computer software via automated processes.

For example computer imaging data may be obtained from one or more imaging techniques, that is, any technique either singly or in combination that can capture and represent, for example, in digital form, the internal anatomy of the human body (the internal anatomical data) of the wearer.

Examples of such techniques include x-ray radiographic, magnetic resonance imaging, medical ultrasonography, medical ultrasound, endoscopy, elastography, tactile imaging, thermography, medical photography, positron emission tomography (PET) and single photon emission computed tomography.

Measurement and recording techniques which are not primarily designed to produce images, such as electroencephalography (EEG), magnetoencephalography (MEG) and electrocardiography (ECG) may also be employed to determine the positioning of the functional elements in the wearable medical device.

When positioned on a wearer's head the wearable device may be headgear. Examples of headgear include, but are not limited to, a helmet, a cap, and a headband.

In one embodiment the wearable device may comprise a sheet of, for example, a thermoplastic material. The wearable device may comprise a thin sheet. The wearable device may comprise an impact-resistant thermoplastic. The wearable device may be aesthetically-pleasing.

The wearable device, for example sheet, may have a thickness between about 0.05 mm and 10 mm, or between about 0.1 mm and about 5 mm, or between about 0.2 mm and about 2 mm. The wearable device, for example sheet, may have a thickness less than 10 mm, or less than 5 mm, or less than 2 mm, or less than 1 mm.

The wearable device, for example sheet, may be flexible. The device, for example sheet, may be elastic. The wearable device, for example sheet, may be curved.

The wearable device may, at least in part, be manufactured using additive manufacturing. It will be appreciated that the practice of applying a heated thermoplastic directly to the external anatomy of the wearer may result in injury and/or suboptimal precision of the contour, with the added potential for discomfort of the wearer and/or suboptimal protection of the anatomy. Advantageously, additive manufacturing provides the ideal method of manufacturing a wearable device based on the anatomical data provided from the one or more imaging devices.

In one embodiment the wearable device may be a full body suit or parts thereof. For example, the wearable device may be a suit or parts thereof manufactured from a suitable elastic material that closely fits the wearer's external anatomy. Non-limiting elastic materials include Spandex®, Lycra® and the like.

The suit, or parts thereof, may comprise one or more functional elements as herein disclosed suitably positioned and/or orientated within the suit, or parts thereof, so as to substantially align with functional elements in one or more internal devices. Accordingly, optimum and advantageous operation of the functional elements may result.

According to the second aspect of the present disclosure the one or more functional elements may be in communication with one or more remote devices. The one or more functional elements may form a network comprising one or more remote devices. Remote devices include, but are not limited to, mobile communication devices such as phones, remote computer servers and the like.

The one or more functional elements may be physically connected to one or more functional elements in a device implanted in the wearer.

The connection may be via suitable male and female connectors. The connection may be temporary or permanent.

The one or more functional elements may be in wireless communication with an internally implanted device.

### Methods of Manufacturing Wearable Medical Devices

In a third aspect of the present disclosure there is provided a method of manufacturing a wearable medical device, in whole or in part, comprising:

(a) determining the contours of the external anatomical surface of a wearer in one or more regions of interest;

(b) designing the wearable device, so that one or more surface regions of the wearable device are contoured to substantially match at least one contour of the one or more regions of interest of the external anatomical surface of a wearer,

(c) manufacturing the device, for example, using additive manufacturing;

(d) positioning one or more functional elements within or on the surface of the manufactured device.

By ‘substantially match’ it may mean that the contoured surface region of the wearable medical device fits an external anatomical surface of a wearer without kinks or overlaps.

In some embodiments the one or more functional elements may be introduced during manufacture of the device.

The one or more contoured surface regions of the wearable medical device may comprise a part or a whole of any one or more of the surfaces of the device that are, in use, in contact with the external anatomical surface of the wearer.

The one or more contoured surface regions of the wearable medical device may be designed so that the device closely fits an external anatomical surface of the wearer.

The one or more contoured surface regions of the wearable medical device may be determined by wearer specific computer imaging data or other processes.

The shape and dimensions of the one or more contoured surface regions of the wearable medical device may be determined using computer software via automated processes, or via human input or by a combination of both

The shape and dimensions of the one or more contoured surface regions of the device may be determined entirely by human input without the use of a computer.

The shape and dimensions of the one or more contoured surface regions of the wearable medical device may be determined by using the techniques of, for example, hand crafting or sculpting, forming or moulding.

In one embodiment the one or more contoured surface regions of the wearable medical device may substantially match the contours of a specific region of the external anatomical surface of the wearer. For example, to substantially match the external anatomical surface of the head, neck, chest, abdomen, pelvis or limbs of the wearer.

In one embodiment the one or more contoured surface regions of the wearable medical device may substantially match the contours of more than one region of the external anatomical surface of the wearer. For example, the wearable medical device may be a body suit or parts thereof.

The wearable medical device may find use in the monitoring and/or modulation of the health of the wearer.

The one or more functional elements may be positioned and/or orientated within the wearable medical device so as to substantially align with specific regions of the external anatomical surface of the wearer.

A preferred method for manufacturing, for example, in the case of a protective headgear, is to use the anatomical data to design and manufacture a template structure, being a structure containing a void corresponding to the precise form and shape of the intended protective headgear, using additive manufacturing techniques. This template structure can be used to form the protective headgear from one or a plurality of plastics or polymers, either in layers, sections or combinations thereof, with the materials including plastics or polymers classified as thermoplastic, thermoset, elastomeric or electrically conductive.

In an embodiment of a patient-specific wearable medical device, and in which the wearer of the patient-specific wearable device also has an implanted medical device, the patient-specific wearable device may be designed so as to integrate one or more electronic devices. Such electronic devices may be configured to measure one or more of physical, chemical, magnetic or optical properties. Such electronic devices may include, but are not limited to, one or more sensors, actuators, energy delivery or transmission devices, energy harvesting devices, energy storage or generation devices and transducers.

In one embodiment the wearable device may sense and quantify environmental parameters, for example, light, sound, electromagnetic radiation, thermal radiation or gravitational forces.

In one embodiment the wearable device may house GPS and satellite communication systems.

In one embodiment the wearable device may allow remote monitoring and feedback.

In a fourth aspect of the present disclosure there is provided a method of manufacturing a wearable medical device, in whole or in part, comprising:

(a) determining the features of the internal anatomical structure of a wearer in one or more regions of interest:

(b) designing the wearable device, so that at least one functional element is positioned within or on said wearable medical device such that, in use, the at least one functional element aligns with an internal anatomical feature of the wearer; and

(c) manufacturing the device, for example, using additive manufacturing.

In some embodiments the one or more functional elements may be introduced during manufacture of the device.

The features of the internal anatomical structure of the wearer may be determined by computer imaging data or other processes.

The features of the internal anatomical structure of the wearer may be determined using computer software via automated processes, or via human input or by a combination of both

The features of the internal anatomical structure of the wearer may be determined entirely by human input without the use of a computer.

In one embodiment the positioning of the functional elements within or on the device may substantially align with a specific region of the internal anatomical structure of the wearer. For example, to substantially match at least a portion of the internal anatomical structure of the brain.

In one embodiment the one or more functional elements of the wearable medical device may be positioned to align with more than one region of the internal anatomical structure of the wearer. For example, the wearable medical device may be a body suit or parts thereof comprising more than one functional element.

The wearable medical device may find use in the monitoring and/or modulation of the health of the wearer.

The one or more functional elements may be positioned and/or orientated within or on the wearable medical device so as to substantially align with specific regions of the internal anatomical structure of the wearer.

A preferred method for manufacturing, for example, in the case of a protective headgear, is to use the anatomical data to design and manufacture a template structure, being a structure containing a void corresponding to the precise form and shape of the intended protective headgear, using additive manufacturing techniques. This template structure can be used to form the protective headgear from one or a plurality of plastics or polymers, either in layers, sections or combinations thereof, with the materials including plastics or polymers classified as thermoplastic, thermoset, elastomeric or electrically conductive.

In an embodiment of a patient-specific wearable medical device, and in which the wearer of the patient-specific wearable device also has an implanted medical device, the patient-specific wearable device may be designed so as to integrate one or more electronic devices. Such electronic devices may be configured to measure one or more of physical, chemical, magnetic or optical properties. Such electronic devices may include, but are not limited to, one or more sensors, actuators, energy delivery or transmission devices, energy harvesting devices, energy storage or generation devices and transducers.

In one embodiment the wearable device may sense and quantify environmental parameters, for example, light, sound, electromagnetic radiation, thermal radiation or gravitational forces.

In one embodiment the wearable device may house GPS and satellite communication systems.

In one embodiment the wearable device may allow remote monitoring and feedback.

### Systems Comprising Wearable and Implanted Devices

In a fifth aspect there is provided a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device externally positioned on said wearer's external anatomy, said wearable device comprising one or more functional elements;

wherein the at least one implanted device and at least one wearable externally positioned device are in communication.

### Systems Comprising Personalized Wearable Medical Devices

In a sixth aspect there is provided a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements; wherein the at least one implanted device and at least one wearable device are in communication; and

wherein the at least one wearable device comprises one or more surfaces, said one or more surfaces having at least one region contoured to substantially match at least one contour of an external anatomical surface of the wearer.

Throughout this specification the term ‘substantially match’ may mean that the contoured surface region of the wearable medical device conforms to an external anatomical surface of a wearer without kinks or overlaps.

The one or more contoured surface regions of the wearable medical device may comprise a part or a whole of any one or more of the surfaces of the device that are, in use, in contact with the external anatomical surface of the wearer.

The one or more contoured surface regions of the wearable medical device may be designed so that the device closely fits an external anatomical surface of the wearer.

The one or more contoured surface regions of the wearable medical device may be determined by wearer specific computer imaging data or other processes.

The shape and dimensions of the one or more contoured surface regions of the wearable medical device may be determined using computer software via automated processes, or via human input or by a combination of both

The shape and dimensions of the one or more contoured surface regions of the device may be determined entirely by human input without the use of a computer.

The shape and dimensions of the one or more contoured surface regions of the wearable medical device may be determined by using the techniques of, for example, hand crafting or sculpting, forming or moulding.

In one embodiment the one or more contoured surface regions of the wearable medical device may substantially match the contours of a specific region of the external anatomical surface of the wearer. For example, to substantially match the external anatomical surface of the head, neck, chest, abdomen, pelvis or limbs of the wearer.

In one embodiment the one or more contoured surface regions of the wearable medical device may substantially match the contours of more than one region of the external anatomical surface of the wearer. For example, the wearable medical device may be a body suit or parts thereof.

The systems may find use in the monitoring and/or modulation of the health of the wearer.

The one or more functional elements may be positioned and/or orientated within the wearable medical device so as to substantially align with specific regions of the external anatomical surface of the wearer.

In a seventh aspect there is provided a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements; wherein the at least one implanted device and at least one wearable device are in communication; and

wherein at least one of said functional elements is positioned within said wearable medical device such that said at least one functional element aligns with a functional element in the implanted device.

Throughout this specification the term ‘aligns’, in the sense of alignment of functional elements in the wearable medical device with functional elements in the implanted device, may mean that the position of the at least one functional element in the wearable medical device is such that its interaction with a functional element in the implanted device is substantially maximized.

Alternatively or additionally, throughout this specification the term ‘aligns’, in the sense of alignment of functional elements in the wearable medical device with functional elements in the implanted device, may be mean that at least one functional element in the wearable medical device and at least one functional element in the implanted device are within proximity such that energy transfer can occur.

Alternatively or additionally, throughout this specification the term ‘aligns’, in the sense of alignment of functional elements in the wearable medical device with functional elements in the implanted device, may be mean that at least one functional element in the wearable medical device and at least one functional element in the implanted device overlap in at least one respective surface by at least 10%, or at least 20%, or at least 30%, or at least 40%, or at least 50%, or at least 60%, or at least 70%, or at least 80%, or at least 90%, or substantially 100%.

Alternatively or additionally, throughout this specification the term ‘aligns’, in the sense of alignment of functional elements in the wearable medical device with functional elements in the implanted device, may mean that at least one functional element in the wearable medical device and at least one functional element in the implanted device are positioned relative to one another so that the angle between a longitudinal axis through the functional element in the wearable device and a longitudinal axis through the functional element in the implanted device is less than 90°, or less than 80°, or less than 70°, or less than 60°, or less than 50°, or less than 40°, or less than 30°, or less than 20°, or less than 10°, or substantially 0°. When the angle is substantially 0°, then the functional element in the wearable medical device and the functional element in the implanted device are substantially parallel with each other.

Alternatively or additionally, throughout this specification the term ‘aligns’, in the sense of alignment of functional elements in the wearable medical device with functional elements in the implanted device, may mean that the centre of at least one functional element in the wearable medical device and the centre of at least one functional element in the implanted device are offset from each other by no more than 10%, or no more than 20%, or no more than 30%, or no more than 40%, or no more than 50%, or no more than 60%, or no more than 70%, or no more than 80%, or no more than 90%, or no more than 100%, relative to the largest dimension of the functional element in the wearable medical device.

The functional elements in the wearable medical device and the implanted medical device may be of any three dimensional shape. Preferred three dimensional shapes include, for example, a sphere, a spheroid, a cube, a rectangular prism, a prism, a triangular prism, a cylinder, a cone, a pyramid, or combinations thereof.

In a non-limiting example, at least one functional element in the wearable device and at least one functional element in the implanted device are positioned to ensure maximum efficiency of, for example, any one or more of energy transfer, communication or system functionality. Accordingly, using wearer specific data from, for example, imaging and/or electrophysiology and/or functionality, additive manufacturing-driven fabrication methods may be utilised as tools to ensure the resulting system remains wearer specific and/or functionally optimized.

For example, for inductive power transfer between pairs of coils, wherein one coil is within a functional element of the wearable device and the other coil is within a functional element of the implanted device the relative angle between the two coils should be kept at approximately, for example, less than 10 degrees to ensure maximal efficiency.

Alignment between functional elements in the implanted device and wearable device may be customised to optimise the function of each wearer's implanted and worn devices by use of a radiant energy scanner to capture internal data and a customised manufacturing process to create a conforming wearable device.

### Systems Comprising Personalized Implanted Devices

In an eighth aspect there is provided a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements;

wherein the at least one implanted device and at least one wearable device are in communication; and wherein the at least one implanted device comprises one or more surfaces, said one or more surfaces having at least one region contoured to substantially match at least one contour of an internal anatomical structure of the wearer.

In a ninth aspect there is provided a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements;

wherein the at least one implanted device and at least one wearable device are in communication;
wherein the at least one implanted device comprises one or more surfaces, said one or more surfaces having at least one region contoured to substantially match at least one contour of an internal anatomical surface of the wearer; and wherein the at least one wearable device comprises one or more surfaces, said one or more surfaces having at least one region contoured to substantially match at least one contour of an external anatomical surface of the wearer.

By ‘substantially match’ it may mean that the contoured surface region of the wearable medical device fits an external anatomical surface of a wearer without kinks or overlaps.

According to any one of the fifth to ninth aspects of the present disclosure examples of functional elements include, but are not limited to, sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures or encasing structures.

According to any one of the fifth to ninth aspects the functional elements in the wearable medical device and the implanted medical device may be of any three dimensional shape. Preferred three dimensional shapes include, for example, a sphere, a spheroid, a cube, a rectangular prism, a prism, a triangular prism, a cylinder, a cone, a pyramid, or combinations thereof.

According to any one of the fifth to ninth aspects of the present disclosure the functional elements may provide means for one or more of sensing, communicating, actuating, delivering or receiving information, delivering or receiving energy, or generating energy.

According to any one of the fifth to ninth aspects of the present disclosure the means for sensing may be one or more of chemical, electrical, physical, optical or magnetic.

According to any one of the fifth to ninth aspects of the present disclosure the means for stimulating may be one or more of chemical, electrical, physical, optical or magnetic.

According to any one of the fifth to ninth aspects of the present disclosure the means for actuating may be one or more of pressure, vacuum or physical deformation.

According to any one of the fifth to ninth aspects of the present disclosure the means for energy generation may be, for example, based on physical movement of the wearer or based on other movements.

According to any one of the fifth to ninth aspects of the present disclosure the functional elements may provide means for monitoring and/or measuring wearer specific parameters and/or non-wearer specific parameters.

Wearer specific parameters include, but are not limited to, temperature, pressure, electrophysiological changes or the concentration and/or nature of one or more chemical species.

Non-wearer specific parameters include, but are not limited to, temperature, pressure, light intensity, electromagnetic radiation, sound, or the concentration and/or nature of one or more chemical species.

The one or more functional elements may provide means for stimulating the wearer.

The one or more functional elements may be in communication with one or more implanted devices. The one or more functional elements may deliver electrical power to, for example, an implanted device.

In one embodiment the functional elements may sense and quantify environmental parameters, for example, light, sound, electromagnetic radiation, thermal radiation or gravitational forces.

In one embodiment the functional elements may comprise GPS and satellite communication systems.

In one embodiment the functional elements may allow remote monitoring and feedback.

The one or more functional elements may be in communication with each other. The one or more functional elements may form a communication network.

The one or more functional elements may comprise one or more electronic components.

The one or more electronic components may provide means for one or more of sensing, stimulating, communicating, actuating, delivering or receiving information, delivering or receiving energy, or generating energy.

According to any one of the fifth to ninth aspects of the present disclosure the shape and dimensions of the one or more contoured surface regions of the wearable medical device may be determined using computer software via automated processes.

For example computer imaging data may be obtained from one or more imaging devices, that is, any device or devices either singly or in combination that can capture and represent, in digital form, the external and/or internal anatomy of the human body (the anatomical data) of the wearer. Examples of such devices include, but are not limited to, Computed Tomography, Magnetic Resonance Imaging, Ultrasound, one or more lasers, one or more digital cameras, and medical ultrasound.

When positioned on a wearer's head the wearable device may be headgear. Examples of headgear include, but are not limited to, a helmet, a cap, and a headband.

Using the anatomical data, the wearable device may be designed to fit the intended wearer by either a person skilled in the use of three dimensional design software, or by using a set of processes automated in software.

In one embodiment the wearable device may comprise a sheet of, for example, a thermoplastic material. The wearable device may comprise a thin sheet. The wearable device may comprise an impact-resistant thermoplastic. The wearable device may be aesthetically-pleasing.

The wearable device, for example sheet, may have a thickness between about 0.05 mm and 10 mm, or between about 0.1 mm and about 5 mm, or between about 0.2 mm and about 2 mm. The wearable device, for example sheet, may have a thickness less than 10 mm, or less than 5 mm, or less than 2 mm, or less than 1 mm.

The wearable device, for example sheet, may be flexible. The device, for example sheet, may be elastic. The wearable device, for example sheet, may be curved.

Where the wearable device is headgear, the device, for example sheet, may be shaped to conform to the temporal, parietal, frontal or occipital bones of a cranium, or combinations and variations thereof. The device, for example sheet, may be shaped to conform to the left or right sides of the cranium.

The wearable device, for example sheet, may be manufactured as a pre-shaped sheet. It may be pre-shaped to conform to the surface contours of the internal or external anatomy of the wearer. That is, the device, for example sheet, is preferably not a flat sheet that has been curved in a single dimension, but rather a sheet that has been manufactured to substantially conform to the contours of internal or external wearer anatomy. For example, the device, for example sheet, may be substantially dome shaped so as to conform to a respective dome shaped contour of wearer anatomy. The device, for example sheet, and the contour of external wearer anatomy may have a substantially hand and glove relationship.

Where the wearable device is headgear, it may be based on the form of a conventional protective helmet such as might be worn by cyclists or other sportspeople, for example rugby or Australian Rules Football players or North American Football (Gridiron) players. In this embodiment, a support structure intended to distribute the force of any impact applied to the helmet to the skull bone around the skull defect may be designed using the anatomical data. A preferred method of manufacturing this support structure would be using additive manufacturing, however it could also be manufactured using subtractive techniques, injection moulding and the like.

In one embodiment, one or more elements of a helmet structure, including inner shells for providing optimal anatomical contour, and outer shells for providing protection, or both, may be designed using the anatomical data of the wearer, and manufactured using additive manufacturing techniques to provide optimal fit, comfort and therefore protection to the brain of a patient, for example a craniectomy patient.

In one embodiment, the wearable device anatomically conforms precisely to the contour of the skull of the intended wearer. The contour of the skull is obtained and determined by using data provided from one or more imaging devices.

The wearable device may, at least in part, be manufactured using additive manufacturing. It will be appreciated that the practice of applying a heated thermoplastic directly to the external anatomy of the wearer may result in injury and/or suboptimal precision of the contour, with the added potential for discomfort of the wearer and/or suboptimal protection of the anatomy. Advantageously, additive manufacturing provides the ideal method of manufacturing a wearable device based on the anatomical data provided from the one or more imaging devices.

In one embodiment the wearable device may be a full body suit or parts thereof. For example, the wearable device may be a suit or parts thereof manufactured from a suitable elastic material that closely fits the wearer's external anatomy. Non-limiting elastic materials include Spandex®, Lycra® and the like.

The suit, or parts thereof, may comprise one or more functional elements as herein disclosed suitably positioned and/or orientated within the suit, or parts thereof, so as to substantially align with specific surface regions of the external anatomy of the wearer and/or substantially align with functional elements in the internal device. Accordingly, optimum and advantageous operation of the functional elements may result.

According to any one of the fifth to ninth aspects of the present disclosure the one or more functional elements may be in communication with one or more remote devices. The one or more functional elements may form a network comprising one or more remote devices. Remote devices include, but are not limited to, mobile communication devices such as phones, remote computer servers and the like.

According to any one of the fifth to ninth aspects of the present disclosure the one or more functional elements may be physically connected to one or more functional elements in a device implanted in the wearer.

The connection may be via suitable male and female connectors. Expand on this. The connection may be temporary or permanent.

According to any one of the fifth to ninth aspects of the present disclosure the one or more functional elements may be in wireless communication with an internally implanted device.

Throughout this specification the functional element in the implanted medical device may comprise one or more electronic components.

Throughout this specification the functional element in the wearable medical device may comprise one or more electronic components.

In one embodiment the implanted device may comprise a sheet of, for example, a thermoplastic material. The implanted device may comprise a thin sheet. The sheets may be made of silicone, polytetrafluoroethylene, polyurethane or other suitable biocompatible materials.

The implanted device, for example sheet, may have a thickness between about 0.05 mm and 10 mm, or between about 0.1 mm and about 5 mm, or between about 0.2 mm and about 2 mm. The implant, for example sheet may have a thickness less than 10 mm, or less than 5 mm, or less than 2 mm, or less than 1 mm.

The implanted device, for example sheet, may be flexible. The implant, for example sheet may be elastic. The implanted device, for example sheet, may be biodegradable. The implanted device, for example, sheet, may be biostable. The implanted device, for example sheet, may be curved.

In one embodiment the implanted device, for example sheet, may be shaped to conform to part of, or the entirety of the frontal, temporal, parietal or occipital lobes of a brain, or of combinations of one or more of these lobes. The implanted device, for example sheet, may be shaped to conform to the left or right sides of the brain, or to a region spanning both sides of the brain.

In one embodiment the implanted device, for example sheet, may be shaped to conform to part of, or the entirety of one or more lobes or surfaces comprising the cerebellum, brainstem and spinal cord.

The implanted device, for example sheet, may be manufactured as a pre-shaped sheet. It may be pre-shaped to conform to the surface contours of internal patient anatomy. That is, the sheet is not a flat sheet that has been curved in a single dimension, but rather a sheet that has been manufactured to substantially conform to the contours of patient anatomy. For example, the implanted device, for example sheet, may be substantially dome shaped so as to conform to a respective dome shaped contour of internal patient anatomy. The sheet and the contour of patient internal anatomy may have a substantially hand and glove relationship.

The implanted device, for example sheet, may be reinforced in certain areas to assist in suture retention; the reinforcement including increasing the material thickness or adding a second, more resilient material, such as woven polyester. Additionally the implanted device, for example sheet, may contain fixation sites, such as perforations, with or without reinforcement, that allow sutures, screws or rivets to be used to secure the sheet to the surrounding hard or soft tissues. Preferably the sheet includes one or more areas of reinforcement adapted to prevent sutures from pulling out or cutting through the sheet.

The implanted device may have other useful properties. For example the implanted device may incorporate one or more pharmaceutical compositions, such as antibiotics or anti-inflammatory compounds. Such agents may be introduced via material porosity or via the incorporation of a second material with such porosity that may or may not be biodegradable at a predetermined rate.

Other advantages include that the implanted device may be thin and flexible and/or elastic, allowing the brain to continue to swell after initial surgery. Further, the implanted device, such as a sheet, may be modifiable which means that it can be trimmed intra-operatively. The implanted device, for example sheet, may be permanent so that it will not resorb or be semi-permanent or slowly resorbable so re-implantation of the bone or another implanted device can be carried out at the best time to promote patient recovery.

The implanted device, for example sheet, may provide a barrier between layers of tissue such that tissue on one side of the implanted device does not adhere to tissue on the other side. Through the choice of materials the implant may avoid tissue ingrowth. For example a silicone or PTFE sheet will generally be resistant to tissue ingrowth and be non-adhesive to tissue.

The implanted device, for example, sheet, may incorporate monitoring probes. Such probes may be incorporated into the implant at the time of manufacture or be attached to specially designed fixtures that allow such probes to be held in position such that they may be secured in direct contact with the underlying brain or associated structures by way of apertures within the sheet. Such probes may perform one or numerous monitoring functions such including, but not limited to the measurement of intracranial pressure, temperature, electroencephalographic activity, electrophysiological activity, blood gas saturation dissolved gas concentration, pH, biochemical species concentration, tissue mechanical properties, tissue optical properties, blood flow, blood velocity, blood rheology, vascular reactivity to pressure, vascular reactivity to biochemical species, vascular endothelial integrity, tissue water content and cellular morphology and microdialysis for biochemical monitoring.

The implanted device, for example sheet, may incorporate therapeutic substances. Such substances may be incorporated into the implanted device at the time of manufacture, for example, by direct binding via porosity of the material or be bound to an incorporated delivery material. Such substances may include antibiotic, anticonvulsant, stem cells or drugs that limit secondary neuronal damage. The substances may be slow release biodegradable substances that release therapeutic agents in a predictable time released way.

The implanted device, for example sheet, may have an enclosed system of channels that allow the circulation of fluid to and from an external device incorporating a positive or negative pressure hydraulic pump. For example such a system could allow the circulation of fluid to modify the temperature of the brain. Such a system could induce regional hypothermia of the brain.

Although described primarily in relation to the human cranium, embodiments of the implanted device, for example sheet, may be suitably fabricated so that it may be used elsewhere in the body including but not limited to the treatment of conditions of the chest, abdomen, pelvis and limbs.

In any one of the fifth to ninth aspects the implanted device includes, but is not limited to, sensory devices, neurological devices, cardiovascular devices, orthopaedic devices, contraceptive devices and cosmetic devices.

The implanted device may be located in the musculoskeletal system, digestive system, respiratory system, urinary system, reproductive system, endocrine system, circulatory system, nervous system or integumentary system.

The implanted device may be positioned within or on or in close proximity to bones, joints, ligaments, tendons, salivary glands, pharynx, esophagus, stomach, small intestine, large intestine, liver, gall bladder, pancreas, trachea, bronchi, lungs, diaphragm, kidneys, ureters, bladder, urethra, ovaries, fallopian tubes, uterus, placenta, testes, prostrate, endocrine glands, heart arteries, veins, lymphatic vessels, lymph nodes, bone marrow, thymus, spleen, brain, brainstem, spinal cord, nerves, sensory organs, mammary glands and subcutaneous tissue.

The implanted device may be positioned within vascular features, physical features, functional features and electrical features or combinations thereof.

Vascular features may be determined by, for example, vascular mapping. Electrical features may be determined by, for example, electroencephalography.

The operation of the at least one implanted device may depend wholly or in part on the operation of the wearable device.

The at least one implanted device may be implanted into the skull, brain, cranial nerves, brainstem of a patient, or any combination thereof.

The at least one implanted device may be diagnostic and/or therapeutic.

The at least one implanted device may be temporary or permanent.

The at least one wearable external device may be positioned on the wearer's head.

The at least one wearable device may be headgear. Examples of headgear include but are not limited to a helmet, a cap and a headband.

The at least one implanted device may customized in one or more surface regions, said customization being based on patient specific computer imaging data, so that said one or more surface regions match one or more contours of said patient's internal anatomy.

The computer imaging data may be obtained from one or more imaging devices, being any device or devices either singly or in combination that can capture and represent, in digital form, the external or internal anatomy of the human body (the anatomical data). Examples of such devices include, but are not limited to Computed Tomography, Magnetic Resonance Imaging, Ultrasound, one or more lasers, one or more digital cameras, and medical ultrasound.

Patients suffering stroke, head injury, epilepsy, brain tumours, and other neurological diseases or disorders that require a neurosurgical procedure, will often require the removal of a portion of skull bone to permit surgical access to the brain.

In some cases, the removed portion of skull bone will not be replaced immediately. Rather, there will be a persistent defect left in the skull for a period of time, determined by the treating surgeon and/or neurologist, until the patient has recovered to the point that the skull defect may be restored.

In one embodiment, a patient who has had a portion of skull bone removed, and in whom the portion of skull bone is not replaced immediately, may receive an implant as hereinbefore described for the monitoring and/or modulation of brain function during the recovery and rehabilitation period.

At the time of removing the skull bone, a neurosurgeon may implant one or more devices as hereinbefore described that are required for the provision of healthcare. These devices may be diagnostic or therapeutic, and may remain in situ temporarily or permanently. Examples of such devices include, but are not limited to, hydrocephalus shunts, recording or stimulating electrodes, optical sensors and/or stimulation devices, pressure monitoring devices, temperature monitoring devices, biochemical sensors and so on.

In an embodiment of the herein disclosed systems wherein the external wearable device is a patient-specific protective headgear, the wearer of the patient-specific protective headgear also has a brain implant. The patient-specific protective headgear is designed with the aim of integrating one or more electronic devices. Such electronic devices could include, but should not be taken to be limited to, one or more sensors, actuators, energy delivery or transmission devices, energy harvesting, storage or generation devices and transducers.

In other cases, the bone will be replaced, or the portion of skull bone removed will be small enough that no restoration of the defect is required.

Optimally, protective headgear is designed and manufactured to fit the wearer, therefore being patient-specific, providing optimal support and protection for the brain exposed by the skull defect.

The at least one implanted device may comprise one or more functional elements. The one or more functional elements may comprise one or more electronic components.

The wearable device may comprise one or more functional elements. The one or more functional elements may comprise one or more electronic components.

The placement of the electronic components in the wearable device may be such that there is alignment between said components and the electronic components in the internal device. This is advantageous as communication between the internal and external wearable device may be improved.

The one or more electronic components in the implanted device may provide means for one or more of sensing, stimulating, communicating, actuating, delivering or receiving information, delivering or receiving energy, or generating energy.

The means for sensing may be one or more of chemical, electrical, physical, optical or magnetic.

The means for stimulating may be one or more of chemical, electrical, physical, optical or magnetic.

The means for actuating may be one or more of pressure, vacuum or deformation.

The means for energy generation may be, for example, physical movement of the patient wearing the device or other movements.

The one or more electronic components may monitor and/or measure patient specific parameters and/or non-patient specific parameters.

Patient specific parameters include, but are not limited to, temperature, pressure, and concentration of one or more species. Non-patient specific parameters include, but are not limited to, temperature, pressure, light intensity, electromagnetic radiation, sound, and one or more chemical species.

The one or more electronic components may provide stimulation to the patient, for example, electrical stimulation to an implanted device. The one or more electronic components may deliver electrical power.

The wearable device may be protective headgear. The protective headgear may comprise a cavity, suitably sized so as to accommodate electronic components. The electronic components may be one or more of sensors, energy delivery devices, energy storage devices.

In a further embodiment, an implant, for example a brain implant, may be powered and communicate to external electronics via one or more interfaces based on electromagnetic, optical, ultrasonic or electrical means.

This may be performed by wireless powering and/or recharging, using techniques such as electromagnetic induction, radio-wave energy harvesting, piezoelectric conversion of ultrasound, or the photoelectric conversion of light.

The internal device may be powered by and communicate to electronics in the external device via one or more interfaces based on one or more of electromagnetic, optical, ultrasonic and electrical means.

The electronics contained within the cavity of the protective headgear may be removable, such that they may continue to operate independently of the protective headgear. Advantageously, this may facilitate the continued operation of the implanted device that depends on said external electronics, in situations during which the wearing of a protective headgear is not practical. In this scenario, the electronics module may be affixed to the head of the patient using a less bulky fixation device, examples of which are given by elastic bands, hook-and-loop fastening systems and the like.

The cavity for housing the external electronics may be accessible from the exterior of the protective headgear, such that inserting and removing the electronics is straightforward and achievable without requiring the wearer of the protective headgear to remove the headgear.

Considering the modalities of sensing that may be employed in a scenario in which an implant operates in concert with external electronics, specific non-limiting examples are now given that illustrate the general concept. Those skilled in the art will appreciate that further combinations of sensing and/or energy delivery devices are possible that, when considered as part of specific embodiments of the present invention, do not deviate from the scope of the invention described herein.

Examples of such devices include, but are not limited to, hydrocephalus shunts, recording or stimulating electrodes, optical sensors and/or stimulation devices, pressure monitoring devices, temperature monitoring devices, biochemical sensors and so on.

An advantage of the present systems are the combination of devices implanted into or onto the skull, brain, cranial nerves, cerebellum, brainstem or any combination thereof, with devices outside the body. The description is not intended to be limiting; those skilled in the art will appreciate that the concepts embodied in the present invention description can be extended to other body parts, other species, i.e. animal or human, and other intended clinical, physiological, electrophysiological, biomechanical or biophysical functions.

In one embodiment the wearable medical device may capture ambient data, such as pressure, temperature, chemical data, sound, light and so forth and, based on one or more of such data, instruct the internal device to, for example, stimulate, measure, adjust, modify, or feedback.

In one embodiment, based on ambient data measurements, the wearable device may instruct the implanted device via electrical stimulation to release therapeutic materials, such as drugs.

In one embodiment, based on ambient data measurements, the wearable device may instruct the implanted device to electrically stimulate the patient's anatomy, for example provide neural stimulation.

In one exemplary embodiment the monitoring of brain pressure, specifically intracranial pressure, is clinically important in the context of preventing death or brain injury due to the development of excessive pressure, itself the cause of disturbances in cerebral blood flow and metabolism, and the physical deformation of brain tissue and/or the pressing of brain tissue against bony structures in the skull.

After craniectomy, intracranial pressure is artificially lowered by the removal of skull bone and exposure of the intracranial contents to atmospheric pressures.

Nonetheless, the pressure inside the skull remains dependent to a large degree on the dynamic physical properties of the brain itself, including cerebrospinal fluid pressure, vascular tone and the degree of tissue swelling. However, the true contribution of these brain tissue properties to the recorded pressure will also be somewhat dependent on atmospheric pressure.

The skilled artisan will appreciate that the measurement of a combination of atmospheric and internal body pressures affords an improvement in the diagnostic utility of pressure monitoring over the standard practice of measuring internal bodily pressure only.

As such, the wearable device may comprise means to measure atmospheric pressure and the implanted device means to measure intracranial pressure. This may provide a more accurate reflection of the absolute values and trends in brain tissue pressure than the intracranial pressure measurement in isolation.

In another exemplary embodiment brain temperature may be influenced by factors including, but not limited to, cerebral blood flow, the presence of infection and the metabolic rate of the brain tissue itself. Akin to the problem of intracranial pressure monitoring, after craniectomy the brain tissue temperature will be influenced to some degree by atmospheric temperature. As such, an accurate measurement of the absolute value of, and trends in, brain temperature in the brain tissue exposed by a craniectomy skull defect, will only be possible by including information about atmospheric temperature as well.

The skilled artisan will appreciate that the measurement of a combination of atmospheric and skin surface and/or internal body temperatures affords an improvement in the diagnostic utility of temperature monitoring over the standard practice of measuring skin surface and/or internal bodily temperature only.

As such, the wearable device may comprise means to measure atmospheric temperature and the implanted device means to measure brain temperature.

Thus in one embodiment, the electronics in the wearable device comprises one or both of pressure and temperature sensors. In another embodiment the brain implanted device comprises one or both of pressure and temperature sensors.

The transmission of data obtained from one or more sensors contained within the implanted device, and one or more sensors contained within the external electronics in the wearable device, may be achieved by one or more of wireless or wired interfaces. For example, data may be transmitted via Bluetooth, WiFi, or radio frequency signals. Thus in one embodiment, the brain implant transmits data to the external electronics via one or more interfaces as previously described. The external electronics then transmits the data, or a reprocessed form of the data, to a further device remote from the patient. This data is then used for healthcare delivery.

In one embodiment of a protective headgear containing integrated electronics, the headgear contains sensors specifically intended to monitor the health of a brain injury or stroke patient. Further, the headgear may contain sensors to measure the degree of brain bulging or retraction within the skull defect introduced by the craniectomy procedure. Measuring such bulging or retraction may provide valuable information regarding the degree of recovery from brain injury, and the scheduling of cranioplasty, being the surgical procedure performed to correct the defect in the skull.

Patients for whom excessive brain retraction occurs, may experience undesirable neurological symptoms. In one embodiment a protective headgear containing integrated electronics to measure the degree of brain bulging or retraction is provided. This offers an improvement to the surgical management of these patients.

The protective headgear may contain integrated electronics to measure the degree of brain bulging or retraction. The devices used to provide the measurement include distance measuring devices employing optical, acoustic, capacitative or electromagnetic techniques. This list of potential techniques is not intended to be limiting, and those of ordinary skill in the art will appreciate that other techniques for measuring distance could be employed.

In another embodiment, the degree of brain bulging or retraction may be measured by a stretch-sensitive device applied directly to the skin surface over the skull defect. In one embodiment of headgear comprising a stretch-sensitive device, the device is connected directly to the protective headgear via a detachable or non-detachable lead. In another embodiment, it communicates wirelessly via one or a plurality of interfaces as previously described.

In any of the hereinbefore disclosed aspects or embodiments the implanted device may comprise a plurality of functional elements. The functional elements may comprise the same or different components.

In any of the hereinbefore disclosed aspects or embodiments the wearable device may comprise a plurality of functional elements. The functional elements may comprise the same or different components.

In any of the hereinbefore disclosed aspects or embodiments the plurality of functional elements in the wearable device may be matched or aligned with a plurality of functional elements in the implanted device.

In any of the hereinbefore disclosed aspects or embodiments the wearable device may comprise a functional element which is aligned with a functional element in the implanted device and the functional element in the implanted device may be in communication with one or more further functional elements in the implanted device. The functional elements may comprise the same or different components.

For example in one embodiment a functional element in the wearable medical device may comprise a transmitting element. The transmitting element may be positioned in the wearable device so as to align with a receiving element in the internal device. The receiving element in the implanted device may be in communication with one or more further functional elements in the implanted device.

In another embodiment the wearable medical device may comprise a functional element which is in communication with a functional element in the implanted device which is in turn in communication one or more further functional elements in the implanted device. The one or more functional elements in the implanted device may be physically connected or wirelessly connected so as to communicate with each other by means of, for example, electromagnetic, optical or acoustic mechanisms.

Accordingly, there is provided a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements;

wherein the at least one implanted device and at least one wearable device are in communication; and
wherein at least one of said functional elements is positioned within said wearable medical device such that said at least one functional element aligns with a functional element in the implanted device;
and wherein the functional element in the implanted device which is aligned with the functional element in the wearable device is physically or wirelessly connected to further functional elements in the implanted device.

There is also provided a system for monitoring the health of a wearer and/or modulating the health of a wearer, said system comprising:

(a) at least one medical device implanted within said wearer's internal anatomy, said at least one implanted device comprising one or more functional elements; and

(b) at least one wearable medical device positioned on said wearer's external anatomy, said wearable device comprising one or more functional elements;

wherein the at least one implanted device and at least one wearable device are in communication.

The at least one wearable device may comprise one or more surfaces, said one or more surfaces having at least one region contoured to substantially match at least one contour of an external anatomical surface of the wearer.

At least one of said functional elements may be positioned within said wearable medical device such that said at least one functional element aligns with a functional element in the implanted device.

At least one functional element within the implanted device may be positioned so as to align with an internal anatomical feature of the wearer.

In any of the hereinbefore disclosed aspects or embodiments the implanted medical device may comprise a plurality of functional elements independently selected from sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures and encasing structures.

In any of the hereinbefore disclosed aspects or embodiments one or more functional elements in the wearable medical device may be in communication with a remote device. The remote device may be a mobile device such as mobile phone or computer. The remote device may be a computer server.

In any of the hereinbefore disclosed aspects or embodiments one or more functional elements in the wearable medical device and/or implanted device may comprise one or more electronic components.

### BRIEF DESCRIPTION OF THE DRAWINGS

FIG. 1 illustrates a wearable medical device according to an embodiment of the present disclosure.

FIG. 2 illustrates a system according to an embodiment of the present disclosure.

FIG. 3 illustrates a system according to an embodiment of the present disclosure.

FIG. 4 illustrates a system according to an embodiment of the present disclosure.

FIG. 5 illustrates a system according to an embodiment of the present disclosure.

FIG. 6 illustrates a system according to an embodiment of the present disclosure.

FIG. 7 illustrates a system according to an embodiment of the present disclosure.

FIG. 8 illustrates a wearable medical device according to an embodiment of the present disclosure.

FIG. 9 illustrates a system according to an embodiment of the present disclosure.

FIG. 10 illustrates a system according to an embodiment of the present disclosure.

FIG. 11 illustrates a system according to an embodiment of the present disclosure.

FIG. 12 illustrates a system according to an embodiment of the present disclosure.

FIG. 13 illustrates a system according to an embodiment of the present disclosure.

FIG. 14 illustrates a system according to an embodiment of the present disclosure.

FIG. 15 illustrates a system according to an embodiment of the present disclosure.

FIG. 16 illustrates a system according to an embodiment of the present disclosure.

FIG. 17 illustrates a wearable medical device according to an embodiment of the present disclosure.

FIG. 18 illustrates a wearable medical device according to an embodiment of the present disclosure.

FIG. 19 illustrates a wearable medical device according to an embodiment of the present disclosure.

FIG. 20 illustrates a wearable medical device according to an embodiment of the present disclosure.

FIG. 21 illustrates a system according to an embodiment of the present disclosure.

### DESCRIPTION OF EXEMPLARY EMBODIMENTS

Before the present devices and/or methods and/or systems are disclosed and described, it is to be understood that unless otherwise indicated this disclosure is not limited to specific devices, components, systems, designs, methods, or the like, as such may vary, unless otherwise specified. It is also to be understood that the terminology used herein is for the purpose of describing particular embodiments only and is not intended to be limiting.

It must also be noted that, as used in the specification and the appended claims, the singular forms ‘a’, ‘an’ and ‘the’ include plural referents unless otherwise specified. Thus, for example, reference to ‘an implant’ may include more than one implant, and the like.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

Disclosed herein are advantageous devices and systems for delivering health care. The devices and systems are based on patient specific information or patient specific anatomy. Accordingly, a patient specific wearable device customized to a patient's external anatomy is provided. Patient specific implantable devices customized to a patient's anatomy are also provided as are systems employing the wearable and internal devices.

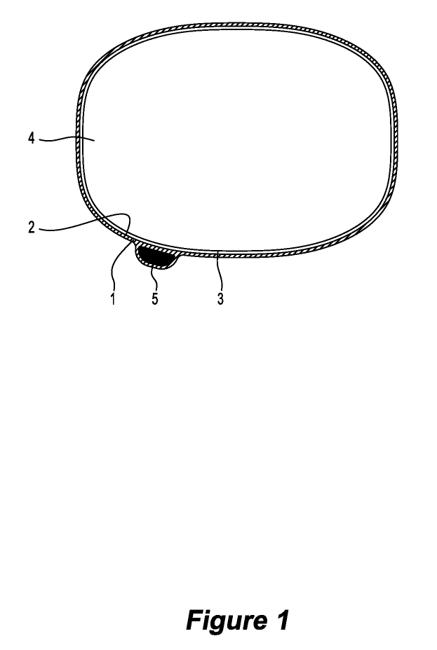

FIG. 1 illustrates a wearable medical device (1) according to an embodiment of the present disclosure having a surface region (2) shaped to substantially match a surface (3) of a wearer's external anatomy. The wearer's internal anatomy is shown as (4). The wearable medical device contains a functional element (5).

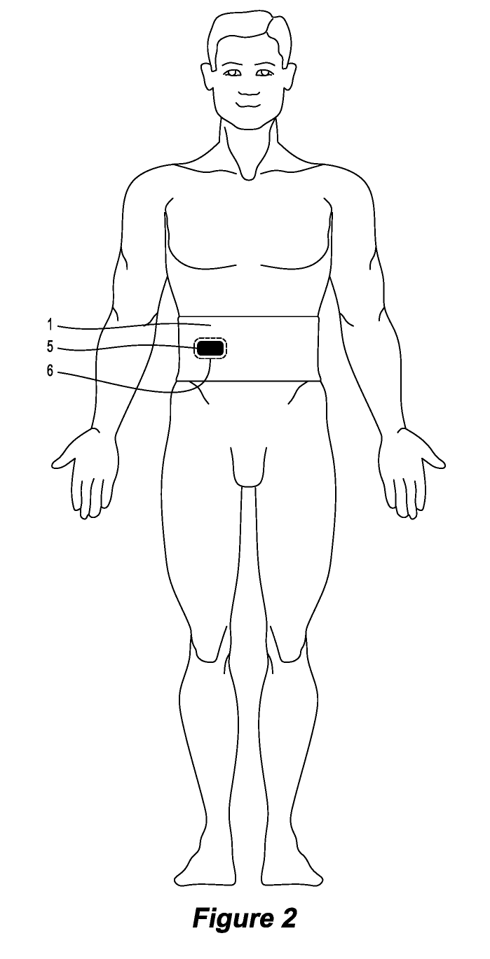

FIG. 2 illustrates a system according to an embodiment of the present disclosure comprising a wearable medical device (1) and an implanted device (6). The wearable medical device is in the form of a waistband and contains a functional element (5).

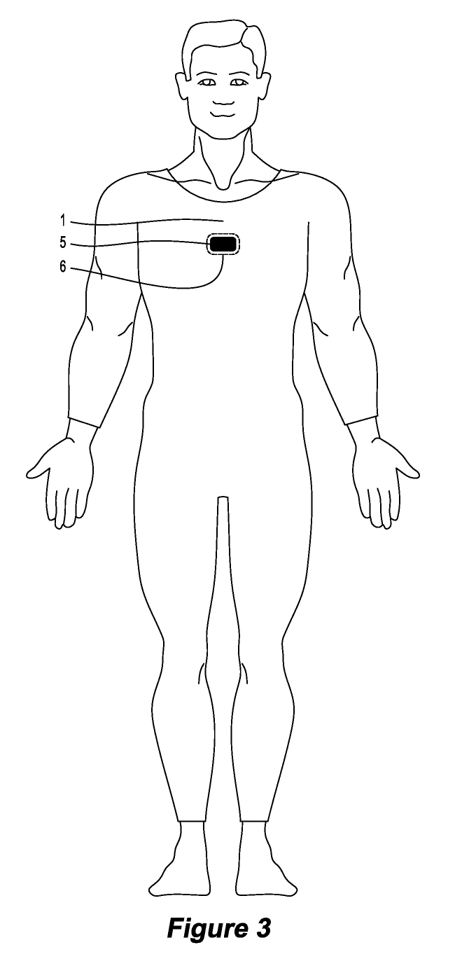

FIG. 3 illustrates a system according to an embodiment of the present disclosure comprising a wearable medical device (1) and an implanted device (6). The wearable medical device is in the form of a body suit and contains a functional element (5).

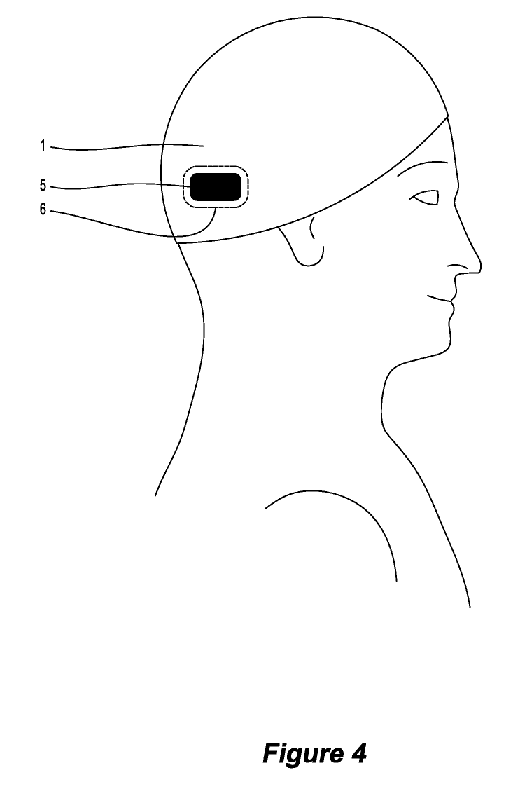

FIG. 4 illustrates a system according to an embodiment of the present disclosure comprising a wearable medical device (1) and an implanted device (6). The wearable medical device is in the form of a head cap and contains a functional element (5)

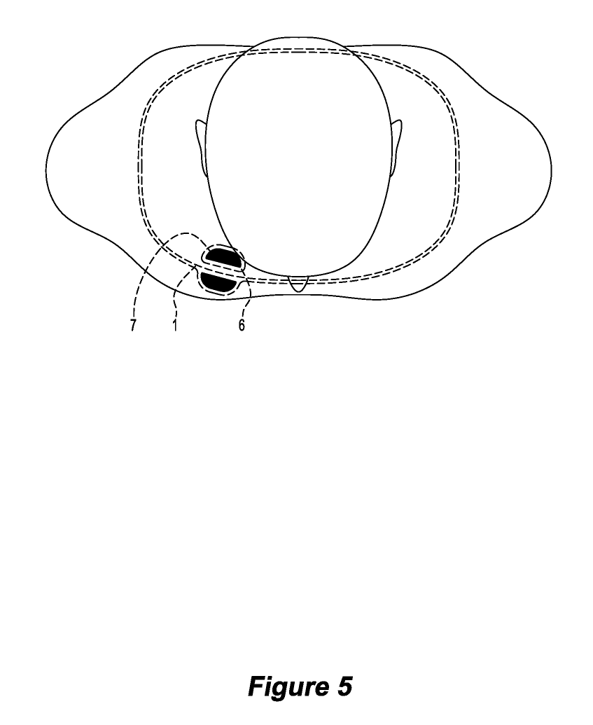

FIG. 5 illustrates a system according to an embodiment of the present disclosure comprising a wearable medical device (1) and an implanted device (6). The wearable medical device is in the form of a waistband and contains functional element (5). The implanted device contains functional element (7).

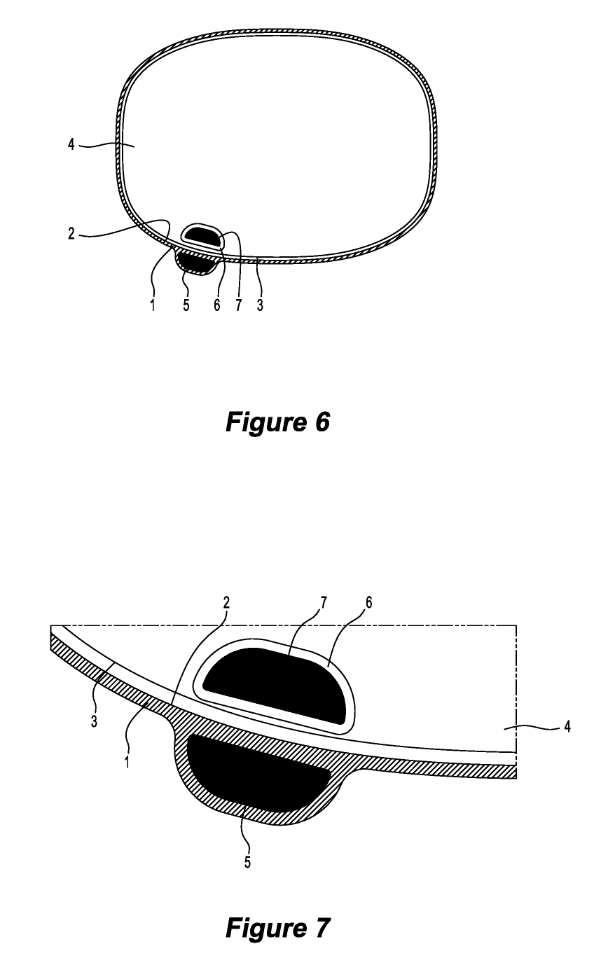

FIG. 6 illustrates a system according to an embodiment of the present disclosure comprising a wearable medical device (1) and an implanted device (6). The wearable medical device has a surface region shaped (2) to substantially match a surface (3) of the wearer's external anatomy. The wearable device contains functional element (5). The implanted device contains functional element (7). The wearer's internal anatomy is shown as (4).

FIG. 7 illustrates a magnification of the system of FIG. 6. Functional element (5) and functional element (7) are aligned so that the overlap in at least one respective surface is substantially 100%. The functional elements are also aligned so that the angle between the longitudinal axis through the functional element in the wearable device and the longitudinal axis through the functional element in the implanted device is substantially 0°. The functional element in the wearable medical device and the functional element in the implanted device are substantially parallel with each other. Additionally, the centre of the functional element in the wearable medical device and the centre of the functional element in the implanted device are offset from each other by no more than 10%, effectively 0%, relative to the largest dimension of the functional element in the wearable medical device.

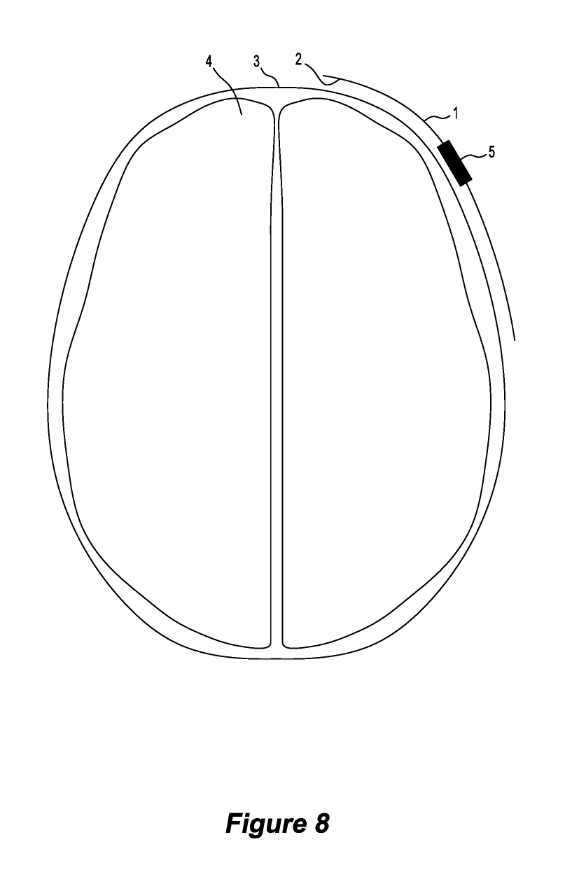

FIG. 8 illustrate a wearable medical device (1) according to an embodiment of the present disclosure having a surface region (2) shaped to match a surface (3) of a wearer's external anatomy. The wearer's internal anatomy, in this embodiment the human brain, is shown as (4). The wearable medical device contains a functional element (5).

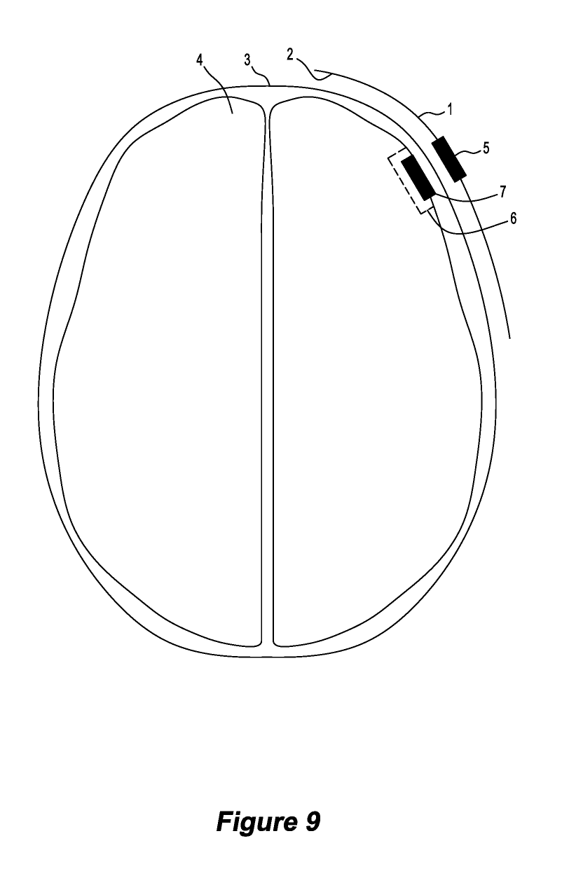

FIG. 9 illustrates a system according to an embodiment of the present disclosure comprising a wearable medical device (1) and an implanted device (6). The wearable medical device has a surface region (2) shaped to substantially match a surface (3) of a wearer's external anatomy and contains functional element (5). The implanted device contains functional element (7). The wearer's internal anatomy, in this embodiment the human brain, is shown as (4). In this embodiment alignment of the functional elements in the devices is optimised to ensure maximum efficiency of operation of the two devices comprising the system. Efficiency may refer to efficiency of power transfer or data communications. Optimisation of alignment may refer to ensuring maximal overlap of the surface area of each device, minimal angulation between the two devices, for example, ensuring they are maximally parallel.

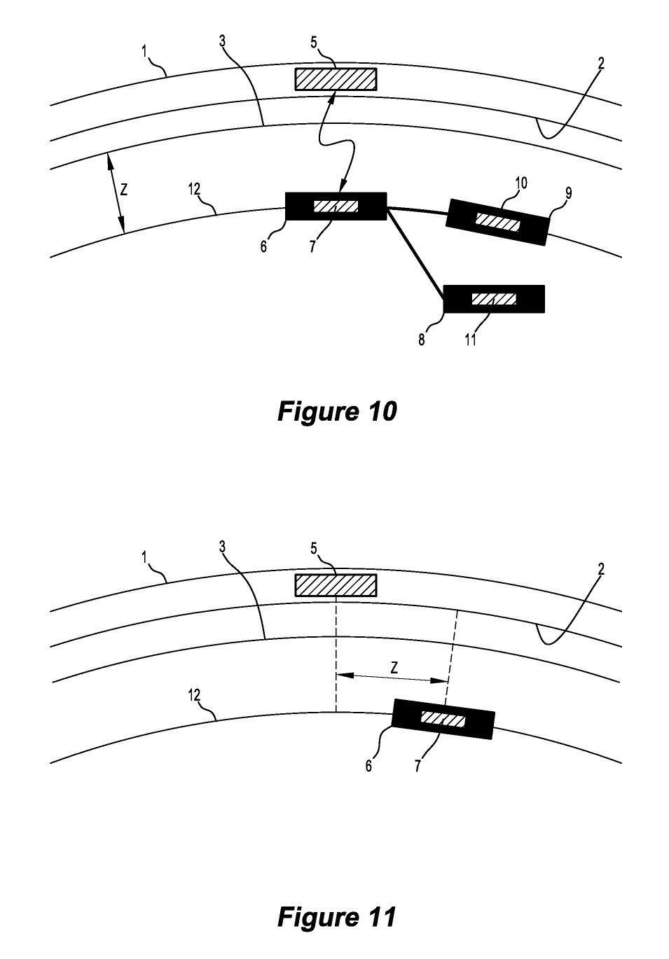

FIG. 10 illustrates a system according to an embodiment of the present disclosure comprising a wearable medical device (1) and an implanted device (6). The wearable medical device has a surface region (2) shaped to substantially match a surface (3) of a wearer's external anatomy and contains functional element (5). The implanted device (6) is implanted in the wearer's tissue (12) at a distance ‘Z’ below the external surface. The device (6) is physically connected or wirelessly connected with implanted devices (8) and (9) which respectively contain functional elements (11) and (10). The implanted devices may form part of a single, larger device. Functional elements (5) and (7) facilitate the transfer of energy between the two as denoted by the double arrow. The energy transferred between the two may be for the purposes of providing power, transferring information or other functions as appropriate to the operation of the device. The alignment between functional elements (5) and (7) is optimised to ensure maximum efficiency of energy transfer between the two.

FIG. 11 illustrates a system in which a functional element (7) in implanted device (6) is misaligned with functional element (5) in wearable device (1). The centre of the functional element in the implanted device is offset from the centre of the functional element in the wearable device by a distance of ‘Z’. Misalignment may occur in any direction. Features (2), (3) and (12) are as defined for FIG. 10. The functional elements are misaligned so that the overlap in at least one respective surface is zero. The functional element in the wearable medical device and the functional element in the implanted device are also misaligned as the centre of the functional element in the wearable medical device and the centre of the functional element in the implanted device are offset from each other (distance ‘Z’) by more than 100%, relative to the largest dimension of the functional element in the wearable medical device.

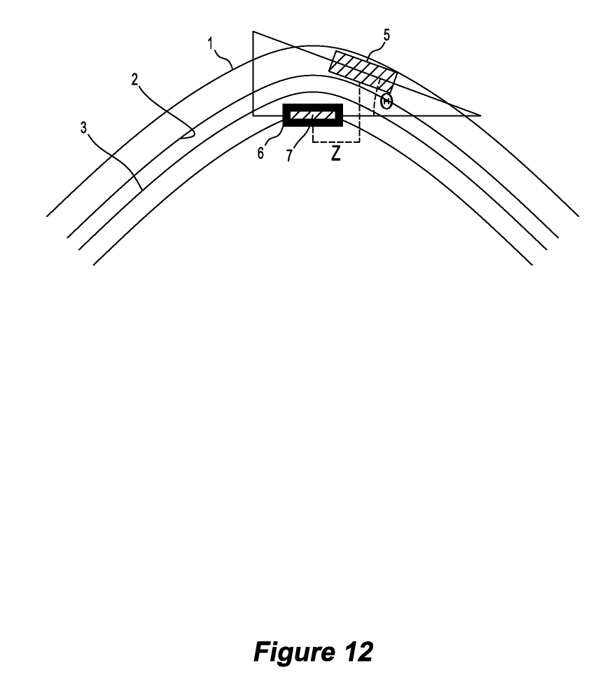

FIG. 12 illustrates a system according to an embodiment of the present disclosure in which a functional element (7) in implanted device (6) is angularly misaligned with functional element (5) in wearable device (1). The centre of the functional element in the implanted device is offset from the centre of the functional element in the wearable device by a distance of ‘Z’ and the direction of a longitudinal axis through functional element (5) is offset from the direction through a longitudinal axis through functional element (7) by an angle theta. Misalignment may occur in any direction. Features (2), (3) and (12) are as defined for FIG. 10.

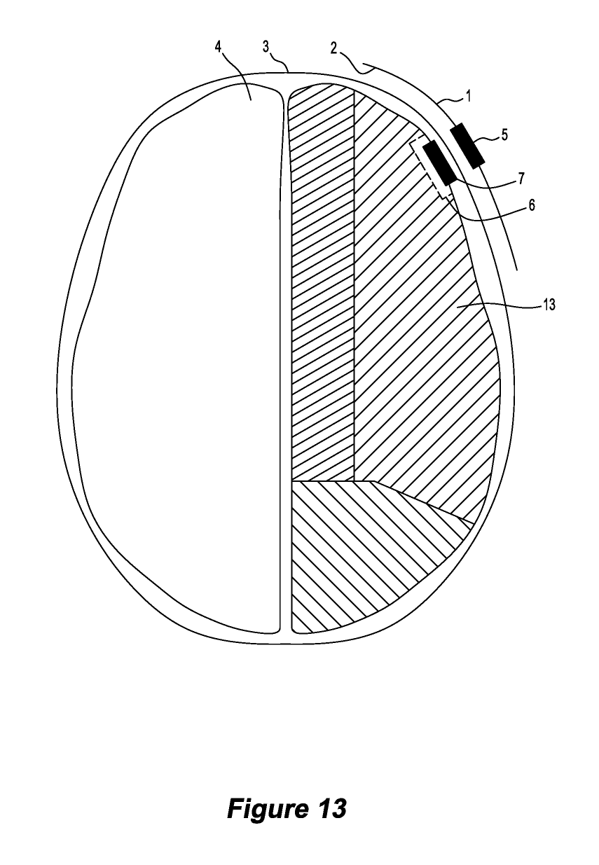

FIG. 13 illustrates a system according to an embodiment of the present disclosure comprising a wearable medical device (1) and an implanted device (6). The wearable medical device has a surface region (2) shaped to substantially match a surface (3) of a wearer's external anatomy and contains functional element (5). The implanted device contains functional element (7). The wearer's internal anatomy, in this embodiment the human brain, is shown as (4). In this embodiment alignment of the functional elements in the devices is optimised to ensure maximum efficiency of operation of the two devices comprising the system. The location of the functional element in the implanted device is such that it overlies or is within a known vascular territory supplying an organ of interest. In the Figure, the vascular territory is the middle cerebral artery territory (13) and the organ of interest is the brain.

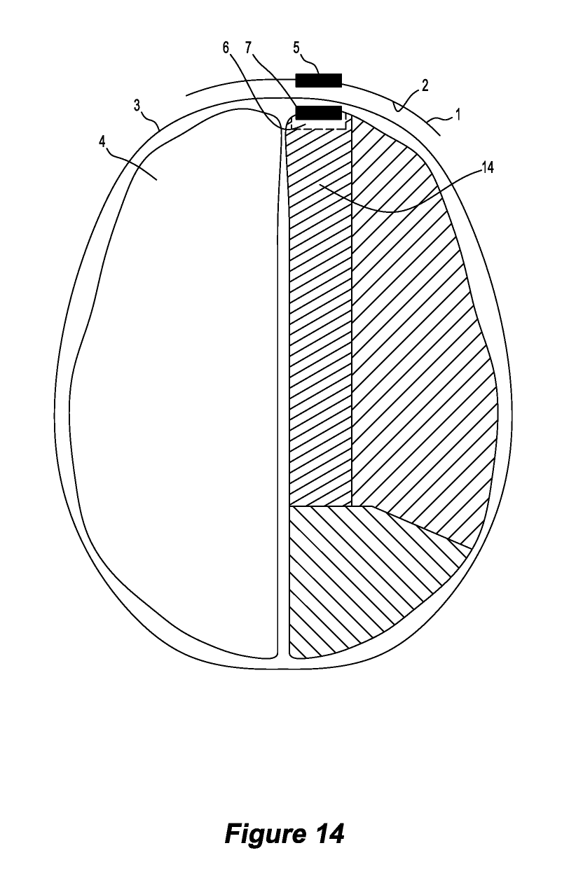

FIG. 14 illustrates a system according to an embodiment of the present disclosure comprising a wearable medical device (1) and an implanted device (6). The wearable medical device has a surface region (2) shaped to substantially match a surface (3) of a wearer's external anatomy and contains functional element (5). The implanted device contains functional element (7). The wearer's internal anatomy, in this embodiment the human brain, is shown as (4). In this embodiment alignment of the functional elements in the devices is optimised to ensure maximum efficiency of operation of the two devices comprising the system. The location of the functional element in the implanted device is such that it overlies or is within a known vascular territory supplying an organ of interest. In the Figure, the vascular territory is the anterior cerebral artery territory (14) and the organ of interest is the brain.

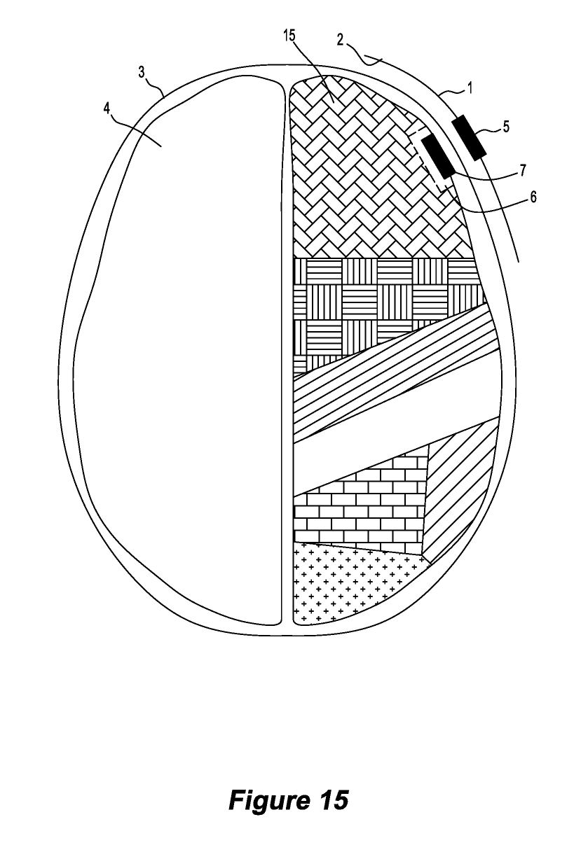

FIG. 15 illustrates a system according to an embodiment of the present disclosure comprising a wearable medical device (1) and an implanted device (6). The wearable medical device has a surface region (2) shaped to substantially match a surface (3) of a wearer's external anatomy and contains functional element (5). The implanted device contains functional element (7). The wearer's internal anatomy, in this embodiment the human brain, is shown as (4). In this embodiment alignment of the functional elements in the devices is optimised to ensure maximum efficiency of operation of the two devices comprising the system. The location of the functional element in the implanted device is such that it overlies or is within a known vascular territory supplying an organ of interest. In the Figure, a particular region (15) of the brain is targeted.

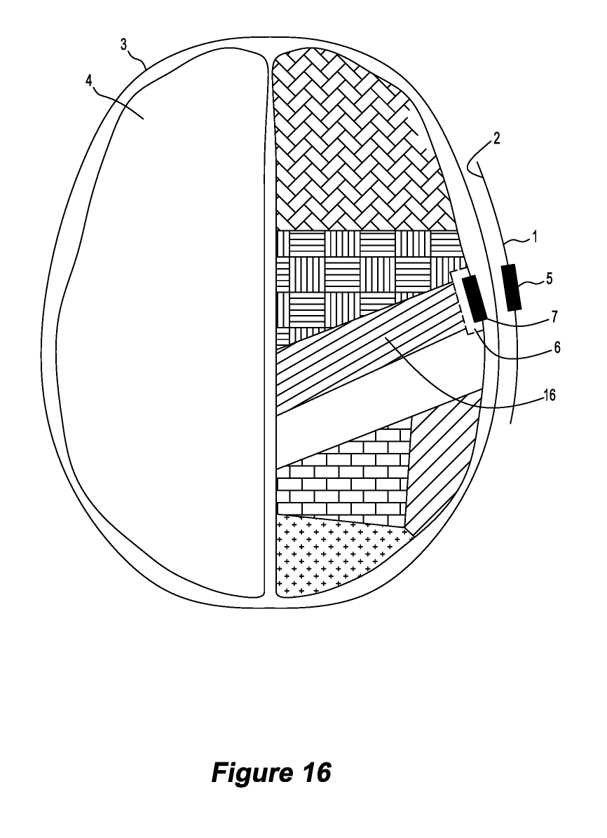

FIG. 16 illustrates a system according to an embodiment of the present disclosure comprising a wearable medical device (1) and an implanted device (6). The wearable medical device has a surface region (2) shaped to substantially match a surface (3) of a wearer's external anatomy and contains functional element (5). The implanted device contains functional element (7). The wearer's internal anatomy, in this embodiment the human brain, is shown as (4). In this embodiment alignment of the functional elements in the devices is optimised to ensure maximum efficiency of operation of the two devices comprising the system. The location of the functional element in the implanted device is such that it overlies or is within a known vascular territory supplying an organ of interest. In the Figure, a particular region (16) of the brain is targeted. This region may be functionally distinct from other regions of the brain.

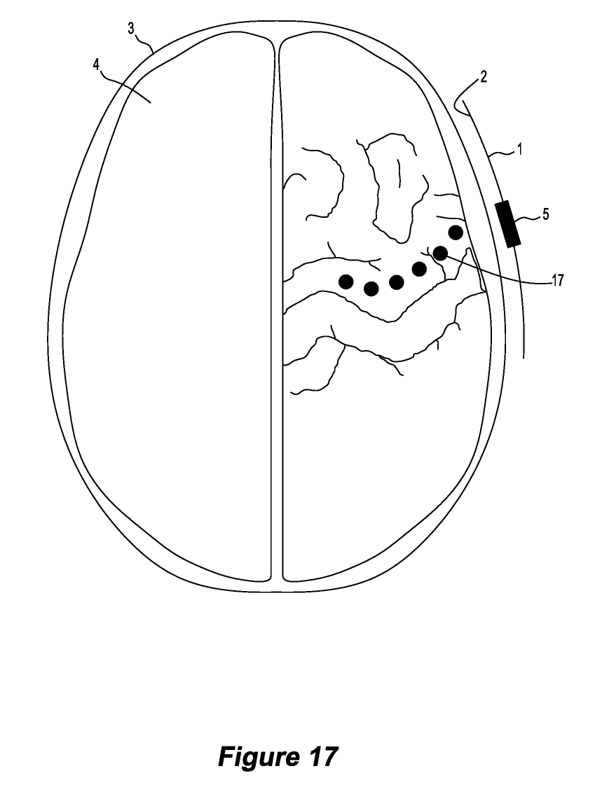

FIG. 17 illustrates a system according to an embodiment of the present disclosure comprising a wearable medical device (1) and an implanted device (17). The wearable medical device has a surface region (2) shaped to substantially match a surface (3) of a wearer's external anatomy and contains functional element (5). The wearer's internal anatomy, in this embodiment the human brain, is shown as (4). In this embodiment anatomical imaging has identified the precentral gyrus and the implanted devices are located within this region.

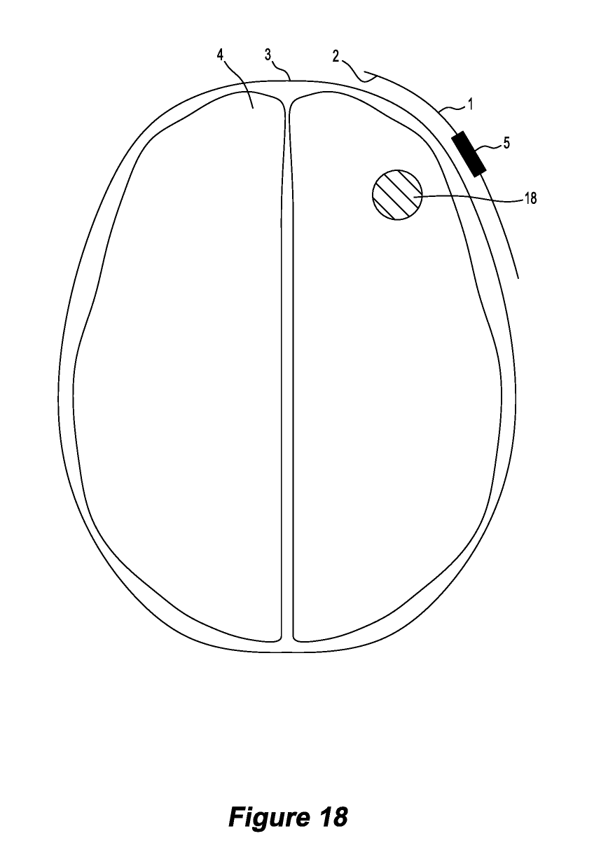

FIG. 18 illustrates a wearable medical device (1) according to an embodiment of the present disclosure having a surface region (2) shaped to substantially match a surface (3) of a wearer's external anatomy. The wearer's internal anatomy is shown as (4). The wearable medical device contains a functional element (5). In this embodiment the region of interest may be identified using anatomical or functional or vascular imaging, for example functional magnetic resonance imaging, magnetic resonance imaging, computed tomographic imaging, ultrasound imaging or digital subtraction angiography. In this non-limiting example, the target organ is the brain. The region of interest is a brain tumour (18).

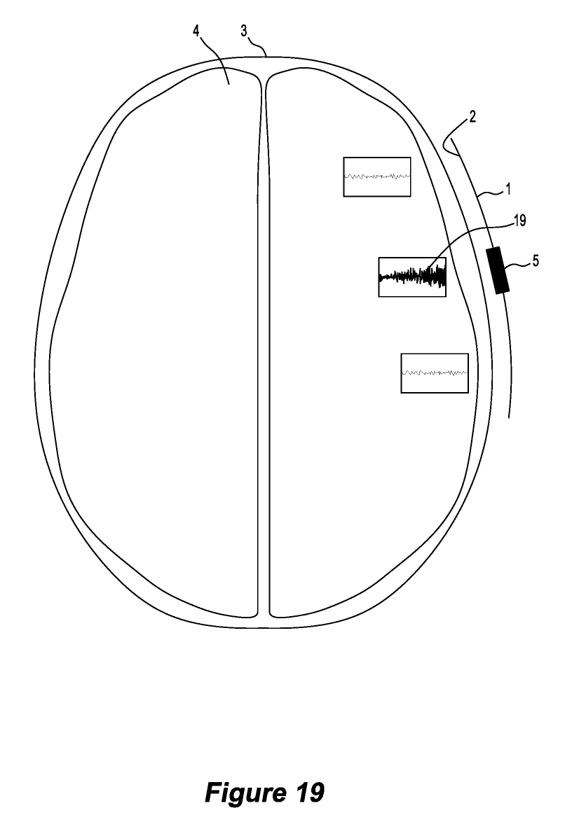

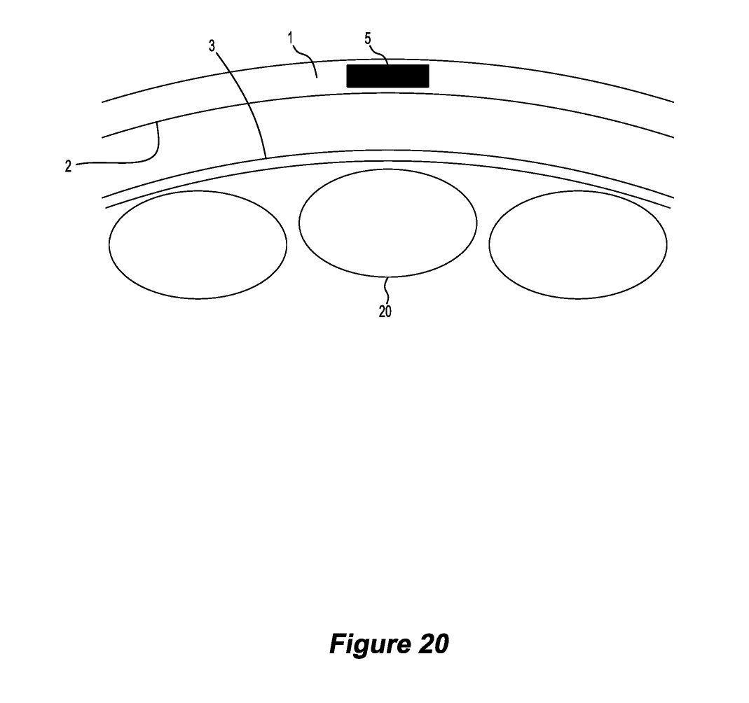

FIG. 19 illustrates a wearable medical device (1) according to an embodiment of the present disclosure having a surface region (2) shaped to substantially match a surface (3) of a wearer's external anatomy. The wearer's internal anatomy is shown as (4). The wearable medical device contains a functional element (5). In this embodiment the region of interest may be identified using electroencephalography (EEG). In this non-limiting example, the target organ is the brain and the region of interest is the source of epileptic seizures, otherwise known as an “epileptic focus” (19).

FIG. 20 illustrates a wearable medical device (1) according to an embodiment of the present disclosure having a surface region (2) shaped to substantially match a surface (3) of a wearer's external anatomy. The wearable medical device contains a functional element (5). In this embodiment the region of interest is the heart (20).

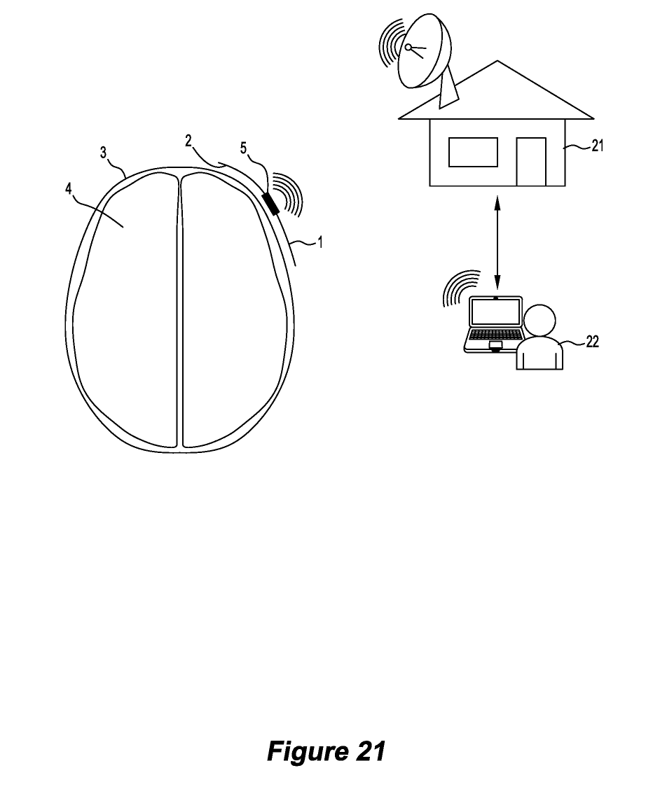

FIG. 21 illustrates a system according to an embodiment of the present disclosure. A wearable device (1) contoured to substantially match one or more external surfaces (3) of the wearer's anatomy, contains one or more functional elements (5). The one or more functional elements may wirelessly communicate, unidirectionally or bidirectionally, with one or more remote devices (21). The one or more remote devices may be a computing device. Examples of such a computing device include, but are not limited to, servers, desktop computers, supercomputers, mobile phones, tablet computers and the like. The computing device (21) may store data transmitted by the one or more functional elements (5). The computing device (21) may run software to analyse and/or visualize the data. The software may require human input to analyse the data. The software may not require human input to analyse the data. The software may be considered “artificially intelligent”. The computing device (21) may communicate information resulting from data analysis and/or human interaction with the one or more functional elements in the wearable device, which may modify their behaviour in response to the information. The wearable device (1) and the computing device 2(1) are therefore a system. The computing device (21) may communicate unidirectionally or bidirectionally with one or more remote devices. The one or more remote devices may be a computing device (22). Examples of such a computing device include, but are not limited to, servers, desktop computers, supercomputers, mobile phones, tablet computers and the like. The computing device (22) may store data transmitted by the computing device (21). The computing device (22) may run software to analyse and/or visualize the data. The software may require human input to analyse the data. The software may not require human input to analyse the data. The software may be considered “artificially intelligent”. The computing device (22) may communicate information resulting from data analysis and/or human interaction with the computing device (21), or it may communicate information directly to the one or more functional elements in the wearable device, which may modify their behaviour in response to the information.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device externally positioned on said wearer's external anatomy, said wearable device comprising one or more functional elements;

wherein the at least one implanted device and at least one wearable externally positioned device are in communication; and
wherein the one or more functional elements in any one of the devices comprise one or more electronic components.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements;

(b) at least one wearable medical device externally positioned on said wearer's external anatomy, said wearable device comprising one or more functional elements; and

(c) at least one device remote from the wearer; said remote device comprising one or more functional elements;

wherein the at least one implanted device and at least one wearable externally positioned device are in communication; and
wherein the at least one of the implanted device or wearable device are in communication with the one or more remote devices.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements;

(b) at least one wearable medical device externally positioned on said wearer's external anatomy, said wearable device comprising one or more functional elements; and

(c) at least one device remote from the wearer; said remote device comprising one or more functional elements;

wherein the at least one implanted device and at least one wearable externally positioned device are in communication;
wherein the at least one of the implanted device or wearable device are in communication with the one or more remote devices; and
wherein the one or more functional elements in any one of the devices comprise one or more electronic components.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements;

wherein the at least one implanted device and at least one wearable device are in communication;
wherein the at least one wearable device comprises one or more surfaces, said one or more surfaces having at least one region contoured to substantially match at least one contour of an external anatomical surface of the wearer; and
wherein the one or more functional elements in any one of the devices comprise one or more electronic components.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements;

(b) at least one wearable medical device positioned on said wearer's external anatomy, said wearable device comprising one or more functional elements; and

(c) at least one device remote from the wearer; said remote device comprising one or more functional elements;

wherein the at least one implanted device and at least one wearable externally positioned device are in communication;
wherein the at least one of the implanted device or wearable device are in communication with the one or more remote devices; and
wherein the at least one wearable device comprises one or more surfaces, said one or more surfaces having at least one region contoured to substantially match at least one contour of an external anatomical surface of the wearer.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements;

(b) at least one wearable medical device positioned on said wearer's external anatomy, said wearable device comprising one or more functional elements; and

(c) at least one device remote from the wearer; said remote device comprising one or more functional elements;

wherein the at least one implanted device and at least one wearable externally positioned device are in communication;
wherein the at least one of the implanted device or wearable device are in communication with the one or more remote devices;
wherein the one or more functional elements in any one of the devices comprise one or more electronic components; and
wherein the at least one wearable device comprises one or more surfaces, said one or more surfaces having at least one region contoured to substantially match at least one contour of an external anatomical surface of the wearer.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements; and

(b) at least one wearable medical device positioned on said wearer's external anatomy, said wearable device comprising one or more functional elements;

wherein the at least one implanted device and at least one wearable device are in communication;
wherein the at least one wearable device comprises one or more surfaces, said one or more surfaces having at least one region contoured to substantially match at least one contour of an internal anatomical surface of a wearer; and
wherein the one or more functional elements in any one of the devices comprise one or more electronic components.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements;

(b) at least one wearable medical device positioned on said wearer's external anatomy, said wearable device comprising one or more functional elements; and

(c) at least one device remote from the wearer; said remote device comprising one or more functional elements;

wherein the at least one implanted device and at least one wearable device are in communication;
wherein the at least one of the implanted device or wearable device are in communication with the one or more remote devices; and
wherein the at least one wearable device comprises one or more surfaces, said one or more surfaces having at least one region contoured to substantially match at least one contour of an internal anatomical surface of a wearer.

In one embodiment the present disclosure provides a system for monitoring the health of a wearer and/or modulating the health of a wearer, said system comprising:

(a) at least one medical device implanted within said wearer's internal anatomy said at least one implanted device comprising one or more functional elements;

(b) at least one wearable medical device positioned on said wearer's external anatomy, said wearable device comprising one or more functional elements; and

(c) at least one device remote from the wearer; said remote device comprising one or more functional elements;

wherein the at least one implanted device and at least one wearable device are in communication;
wherein the at least one of the implanted device or wearable device are in communication with the one or more remote devices;
wherein the one or more functional elements in any one of the devices comprise one or more electronic components; and
wherein the at least one wearable device comprises one or more surfaces, said one or more surfaces having at least one region contoured to substantially match at least one contour of an internal anatomical surface of a wearer.

While the foregoing description has focused on certain parts of the human anatomy, it is contemplated that the devices and systems described herein may find use in a wide range of applications, where wearable and implantable devices are required. Thus, where it is desired to deliver healthcare to other parts of the anatomy, the devices and systems of the present disclosure may be used.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the above examples are put forth so as to provide those skilled in the art with a complete disclosure and description of how to make and use the disclosed devices, and are not intended to limit the scope of the disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All documents cited are herein fully incorporated by reference for all jurisdictions in which such incorporation is permitted and to the extent such disclosure is consistent with the description of the present disclosure.

1-43. (canceled)

44. A system for monitoring the health of a wearer and/or modulating the health of a wearer, said system comprising:
(a) at least one medical device implanted within said wearer's internal anatomy, said at least one implanted device comprising one or more functional elements; and
(b) at least one wearable medical device positioned on said wearer's external anatomy, said wearable device comprising one or more functional elements;
wherein the at least one implanted device and at least one wearable device are in communication.

45. A system according to claim 44, wherein the at least one wearable device comprises one or more surfaces, said one or more surfaces having at least one region contoured to substantially match at least one contour of an external anatomical surface of the wearer.

46. A system according to claim 45, wherein the one or more contoured surface regions of the wearable medical device are obtained from imaging techniques including computed tomography, magnetic resonance imaging, ultrasound, one or more lasers, digital photography, medical ultrasound and combinations thereof.

47. A system according to claim 44, wherein at least one of said functional elements is positioned within said wearable medical device such that said at least one functional element aligns with a functional element in the implanted device.

48. A system according to claim 44, wherein at least one functional element within the implanted device is positioned so as to align with an internal anatomical feature of the wearer.

49. A system according to claim 44, wherein at least one functional element in the wearable medical device and at least one functional element in the implanted device are within proximity such that energy transfer can occur.

50. A system according to claim 44, wherein at least one functional element in the wearable medical device and at least one functional element in the implanted device overlap in at least one respective surface by at least 10%, or at least 20%, or at least 30%, or at least 40%, or at least 50%, or at least 60%, or at least 70%, or at least 80%, or at least 90%, or substantially 100%.

51. A system according to claim 44, wherein at least one functional element in the wearable medical device and at least one functional element in the implanted device are positioned relative to one another so that the angle between a longitudinal axis through the functional element in the wearable device and a longitudinal axis through the functional element in the implanted device is less than 90°, or less than 80°, or less than 70°, or less than 60°, or less than 50°, or less than 40°, or less than 30°, or less than 20°, or less than 10°, or substantially 0°.

52. A system according to claim 44, wherein the centre of at least one functional element in the wearable medical device and the centre of at least one functional element in the implanted device are offset from each other by no more than 10%, or no more than 20%, or no more than 30%, or no more than 40%, or no more than 50%, or no more than 60%, or no more than 70%, or no more than 80%, or no more than 90%, or no more than 100%, relative to the largest dimension of the functional element in the wearable medical device.

53. A system according to claim 44, wherein the position of the one or more functional elements in the wearable medical device is aligned with one or more functional elements in the implanted device by determining the position of the one or more functional elements in the implanted device by x-ray radiography, magnetic resonance imaging, medical ultrasonography, medical ultrasound, endoscopy, elastography, tactile imaging, thermography, medical photography, positron emission tomography (PET), single-photon emission computed tomography (SPECT), electroencephalography (EEG), magnetoencephalography (MEG), electrocardiography (EGG) and combinations thereof.

54. A system according to claim 44, wherein the implanted device is positioned within the wearer's internal anatomy so that the one or more functional elements in the implanted device aligns with internal anatomical features of the wearer and wherein said internal anatomical features of the wearer are determined by x-ray radiography, magnetic resonance imaging, medical ultrasonography, medical ultrasound, endoscopy, elastography, tactile imaging, thermography, medical photography, positron emission tomography (PET), single photon emission computed tomography, electroencephalography (EEG), magnetoencephalography (MEG) electrocardiography (ECG) and combinations thereof.

55. A system according to claim 44, wherein the functional element is selected from the group consisting of temperature sensors, biochemical sensors, mechanical sensors, electrical sensors, ultrasonic sensors, optical sensors, fluid/pressure control elements, stimulating electrodes, stimulating electromagnets, light sources, ultrasonic emitters, power generation elements, energy delivery or receiving elements and combinations thereof.

56. A system according to claim 55, wherein the functional elements provide means for one or more of sensing, communicating, actuating, delivering or receiving information, delivering or receiving energy, or generating energy.

57. A system according to claim 56, wherein the functional elements provide means for monitoring and/or measuring wearer specific parameters and/or non-wearer specific parameters.

58. A system according to claim 57, wherein the wearer specific parameters include, temperature, pressure, electrophysiological changes or the concentration and/or nature of one or more chemical species and combinations thereof.

59. A system according to claim 44, wherein the one or more functional elements are in communication with one or more devices remote from the wearer.

60. A system according to claim 44, wherein, independently the wearable device and the implanted device comprises a sheet of, for example, a thermoplastic material.

61. A system according to claim 60, wherein the sheet has a thickness between about 0.05 mm and 10 mm, or between about 0.1 mm and about 5 mm, or between about 0.2 mm and about 2 mm.

62. A system according to claim 44, wherein at least one functional element in the implanted device is in communication with one or more further functional elements in the implanted device.

63. A system according to claim 47, wherein at least one functional element in the implanted device is in communication with one or more functional elements in one or more further implanted devices in the wearer.
